(19) United States
(12) Patent Application Publication
(10) Pub. No.: US 2007/0211845 A1
(43) Pub. Date: Sep. 13, 2007

(54) X-Ray CT Apparatus

(76) Inventors: Akihiko Nishide, Tokyo (JP); Akira Hagiwara, Tokyo (JP); Kotoko Morikawa, Tokyo (JP)

Correspondence Address: Thomas M. Fisher;Fisher Patent Law Group LLC, 700 6th Street NW, Hickory, NC, 28601, US

(21) Appl. No.: 11/620,665

(22) Filed: Jan. 6, 2007

(30) Foreign Application Priority Data
Mar. 9, 2006 (JP) 2006-063765

(51) Int. Cl. H05G 1/60; A61B 6/00; G01N 23/00; G21K 1/12

(52) U.S. Cl. 378/4

(57) ABSTRACT

X-ray CT apparatus includes an x-ray data collecting device for collecting x-ray projection data transmitted by a subject positioned between an x-ray generating device and a multi-row x-ray detector, while rotating said x-ray generating device and said multi-row x-ray detector around a rotation center positioned in-between, an image reconstructing device for performing image reconstruction from the projection data collected from the x-ray data collecting device, an image display device for displaying a tomogram obtained by image reconstruction, and a scanning condition setting device for setting various scanning conditions of tomography scanning. The x-ray data collecting device is operable for variable-pitch helical scanning which x-ray projection data of the subject on a scanning table is collected by moving the scanning table while varying the speed relative to a scanning gantry in a z direction perendicular to an xy plane which is the rotating plane of the x-ray generating device and the two-dimensional x-ray area detector, and of which starting of the x-ray data collection and starting of the scanning table movement relative to the scanning gantry and/or stopping of the x-ray data collection and stopping of the scanning table movement relative to the scanning gantry are asynchronously executed.

### CROSS REFERENCE TO RELATED APPLICATIONS

This application claims the benefit of Japanese patent application number 2006-063765 filed Mar. 9, 2006.

### BACKGROUND OF THE INVENTION

The present invention relates to an x-ray CT (Computed Tomography) apparatus for medical use or an x-ray CT apparatus for industrial use, to improving the picture quality of imaging methods.

Conventionally, in an x-ray CT apparatus using a multi-row x-ray detector x-ray CT apparatus or a two-dimensional x-ray area detector represented by a flat panel x-ray detector, data were collected in a constant speed part in constant speed helical scanning as shown in FIG. 16 (see JP-A No. 2004-073360 for instance). As a result, there were such wastes and problems: data collection had to wait until the speed of the cradle on the scanning table reached a certain level; a run-up distance was needed until the speed of the cradle reached a certain level; accordingly a region in which scanning was impossible in the travel distance of the cradle as long as this run-up distance; and the scannable region was narrowed or the start of scanning had to wait as long as the time taken by acceleration in the run-up.

For this reason, variable-pitch helical scanning to collect x-ray data even in the z-directional accelerating region at the time of starting the scanning table for helical scanning or in the z-directional decelerating region at the time of ending the operation was called for, but it was difficult to secure the uniformity of picture quality in the z direction in the accelerating region and the decelerating region of variable-pitch helical scanning.

However, in the multi-row x-ray detector x-ray CT apparatus or the two-dimensional x-ray area detector represented by a flat panel x-ray detector, as the cone angle of the x-ray cone beam becomes greater, the table speed becomes DP/t (mm/sec) wherein the width of the detector in the z direction is represented by D (mm), the scanning time per rotation by t (sec/rotation) and the pitch of helical scanning by p. Wherein, stands for multiplication and * represents the convolution operator.

A tendency of existing x-ray CT apparatuses is for the detector width D in the z direction to increase and for the scanning speed to become faster, namely for the scanning time per rotation t to become shorter. Also, the permissible range of the helical pitch p of helical scanning is widened by the three-dimensional image reconstruction, which permits a greater helical pitch, and a greater helical pitch p enables the table speed D p/t(m/sec) to become faster. As a consequence, the run-up distance also tends to be elongated by the increased table speed, and the scannable region is apt to be narrowed.

Thus, if the width of the x-ray detector in the z direction increases or if the relative speed between the scanning table and the x-ray detector becomes faster in the future, where the length of the scanning table is to be fully utilized to shorten the unimaginable range of the scanning table, variable-pitch helical scanning to collect x-ray data in the accelerating region and the decelerating region is required. However, this involved the problem that there arose a difference between the picture quality of tomograms in the constant speed region of helical scanning and the picture quality of tomograms in the accelerating region and the decelerating region. For this reason, variable-pitch helical scanning has not been used.

Therefore, the methods and apparatus described below provide an x-ray CT apparatus capable of securing the uniformity of picture quality in the z direction of tomograms consecutive in the z direction, in variable-pitch helical scanning or helical shuttle scanning by the x-ray CT apparatus having a multi-row x-ray detector or a two-dimensional area x-ray detector of a matrix structure, represented by a flat panel x-ray detector.

### BRIEF DESCRIPTION OF THE INVENTION

In one aspect, an x-ray CT apparatus is provided. The apparatus includes an x-ray data collecting device for collecting x-ray projection data transmitted by a subject positioned between an x-ray generating device and a multi-row x-ray detector, while rotating said x-ray generating device and said multi-row x-ray detector around a rotation center positioned in-between, an image reconstructing device for performing image reconstruction from the projection data collected from the x-ray data collecting device, an image display device for displaying a tomogram obtained by image reconstruction, and a scanning condition setting device for setting various scanning conditions of tomography scanning. The x-ray data collecting device is operable for variable-pitch helical scanning which x-ray projection data of the subject on a scanning table is collected by moving the scanning table while varying the speed relative to a scanning gantry in a z direction perpendicular to an xy plane which is the rotating plane of the x-ray generating device and the two-dimensional x-ray area detector, and of which starting of the x-ray data collection and starting of the scanning table movement relative to the scanning gantry and/or stopping of the x-ray data collection and stopping of the scanning table movement relative to the scanning gantry are asynchronously executed.

In another aspect, a method includes changing a helical pitch during z-direction velocity changes of a moving gantry to obtain substantially uniform image quality in a plurality of reconstructed images.

### BRIEF DESCRIPTION OF THE DRAWINGS

FIG. 1 is a block diagram of an x-ray CT apparatus in one mode for carrying out the invention.

FIG. 2 is a diagram illustrating an x-ray generating device (x-ray tube) and a multi-row x-ray detector as viewed on the xy plane.

FIG. 3 is a diagram illustrating an x-ray generating device (x-ray tube) and a multi-row x-ray detector as viewed on the xy plane.

FIG. 4 is a flow chart showing the flow of imaging a subject.

FIG. 5 is a flow chart outlining the operation of the x-ray CT apparatus pertaining to one mode for carrying out the invention.

FIG. 6 is a flow chart showing details of pre-treatments.

FIG. 7 is a flow chart showing details of three-dimensional image reconstruction processing.

FIG. 8 are conceptual diagrams showing a state of projecting lines on a reconstruction region in the x-ray transmitting direction.

FIG. 9 is a conceptual diagram showing a state of projecting lines on a reconstruction region in the x-ray transmitting direction.

FIG. 10 is a conceptual diagram showing lines projected on detector faces.

FIG. 11 is a conceptual diagram showing a state of projecting projection data Dr(view, x, y) on the reconstruction region.

FIG. 12 is a conceptual diagram showing back-projection pixel data D2 of pixels on the reconstruction region.

FIG. 13 is a diagram illustrating a state in which back-projection data D3 are obtained by subjecting the back-projection pixel data D2 to all-view addition pixel by pixel.

FIG. 14 is a conceptual diagram showing a state of projecting lines on a circular reconstruction region in the x-ray transmitting direction.

FIG. 15 is a diagram showing a scanning condition input screen for the x-ray CT apparatus.

FIG. 16 is a diagram illustrating the range in which helical scanning is possible.

FIG. 17 is a diagram showing a case of constant speed helical scanning.

FIG. 18 is a diagram showing a case of variable speed helical scanning.

FIG. 19 is a diagram showing a case in which the data collection line is inclined.

FIG. 20 is a flow chart of Implementation Example 1 of variable-pitch helical scanning.

FIG. 21 is a diagram showing the operation of Implementation Example 1 of variable-pitch helical scanning.

FIG. 22 is a flow chart of Implementation Example 2 of variable-pitch helical scanning.

FIG. 23 is a diagram showing the operation of Implementation Example 2 of variable-pitch helical scanning.

FIG. 24 is a diagram showing filter convolution of projection data in the z direction.

FIG. 25 is a diagram showing filter convolution of image space in the z direction.

FIG. 26 is a diagram showing processing of processing data view.

FIG. 27 is a table comparing the advantages and disadvantages of the method of convoluting the z-directional filter on projection data and the method of convoluting the z-directional filter on image space.

FIG. 28 is a diagram showing inconsistencies in the z-directional filter width of projection data.

FIG. 29 is a diagram showing an inconsistency-free image space z-directional filter.

FIG. 30 is a diagram showing projection data view weighting by one turn or more.

FIG. 31 is a table of projection data space z filter coefficients and image space z filter coefficients in variable-pitch helical scanning.

FIG. 32 is a diagram showing the operation of shuttle mode variable-pitch helical scanning.

FIG. 33 is a diagram showing the operation of variable-pitch helical scanning.

FIG. 34 is a diagram showing the positional relationship between the data collection line and the tomogram in conventional scanning (axial scanning) or cine-scanning.

FIG. 35 is a diagram showing the positional relationship between the data collection line and the tomogram in helical scanning.

FIG. 36 is a diagram showing the positional relationship among a view a and a view b opposing each other and a tomogram

FIG. 37 is a diagram showing the total imaging range and partial imaging ranges.

FIG. 38 is a diagram showing a range in which tomogram image reconstruction is possible in Implementation Example 1.

FIG. 39 is a diagram showing a range in which tomogram image reconstruction is possible in Implementation Example 2.

FIG. 40 is a diagram showing the relative actions of the x-ray data collection line and the subject by two-way variable-pitch helical scanning in the z direction (equivalent to 1.5 legs).

FIG. 41(a) is a diagram showing the time resolution at different points in two-way helical shuttle scanning.

FIG. 41(b) is a diagram showing the time resolution at different points in one-way helical shuttle scanning.

FIG. 42 is a diagram showing Example 1 of the relationship among the helical pitch, the number of turns of data used and the x-ray tube current of two-way variable-pitch helical scanning or helical shuttle scanning back and forth in the z direction.

FIG. 43 is a diagram showing Example 2 of the relationship among the helical pitch, the number of turns of data used and the x-ray tube current of two-way variable-pitch helical scanning or helical shuttle scanning back and forth in the z direction.

FIG. 44 is a diagram showing Example 3 of the relationship among the helical pitch, the number of turns of data used and the x-ray tube current of two-way variable-pitch helical scanning or helical shuttle scanning back and forth in the z direction.

FIG. 45 is a flow chart of an x-ray automatic exposure function which determines the x-ray tube current in consideration of the quantity of data to be used in image reconstruction.

### DETAILED DESCRIPTION OF THE INVENTION

The present invention will be described in further detail with reference to modes for carrying it out illustrated in drawings. Incidentally, this is nothing to limit the invention.

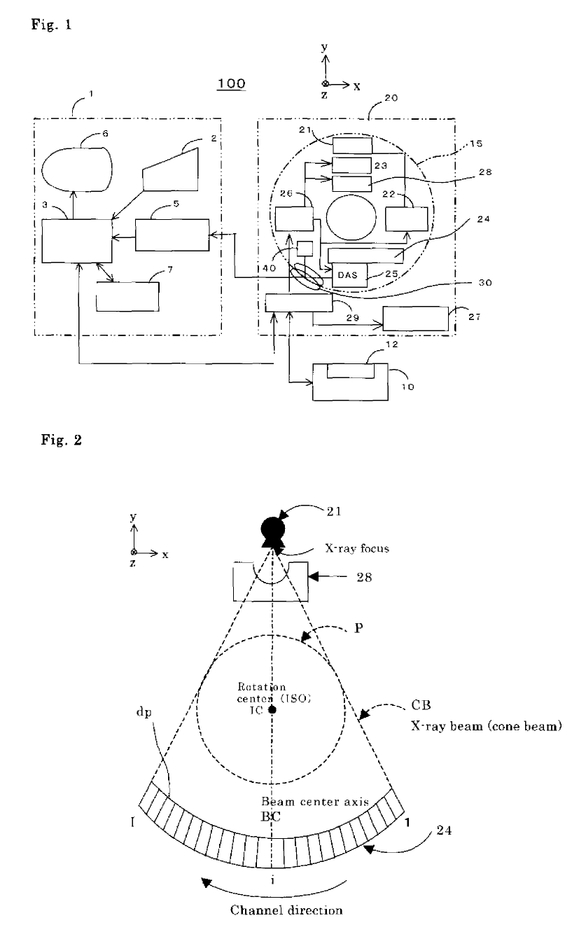

FIG. 1 is a configurational block diagram of an x-ray CT apparatus in one mode for carrying out the invention. This x-ray CT apparatus 100 is equipped with an operation console 1, a scanning table 10 and a scanning gantry 20.

The operation console 1 is equipped with an input device 2 for accepting inputs by the operator, a central processing unit 3 for executing pre-treatments, image reconstruction processing, post-treatments and the like, a data collecting buffer 5 for collecting projection data collected by the scanning gantry 20, a monitor 6 for displaying tomograms reconstructed from projection data obtained by pre-treating x-ray detector data, and a storage unit 7 for storing programs, x-ray detector data, projection data and x-ray tomograms.

Imaging conditions are inputted through this input device 2 and stored in the storage unit 7. FIG. 15 shows an example of input screen of scanning conditions.

The scanning table 10 is equipped with a cradle 12 which places in and out a subject mounted therewith, through the opening of the scanning gantry 20. The cradle 12 is lifted, lowered and moved along the table line by a motor built into the scanning table 10.

The scanning gantry 20 is equipped with an x-ray generating device 21, an x-ray controller 22, a collimator 23, a beam forming x-ray filter 28, a multi-row x-ray detector 24, a DAS (Data Acquisition System) 25, a rotary unit controller 26 for controlling the x-ray generating device 21 and others rotating around the body axis of the subject, and a regulatory controller 29 for exchanging control signals and the like with the operation console 1 and the scanning table 10. The beam forming x-ray filter 28 is an x-ray filter which is the least in filter thickness in the direction of x-rays toward the rotation center, which is the center of imaging, and increases in filter thickness toward the peripheries to enable more of x-rays to be absorbed. For this reason, exposure of the body surface of a subject whose sectional shape is close to a circle or an oval to radiation can be reduced. Further, the scanning gantry 20 can be inclined ahead of or behind in the z direction by approximately ±30 degrees by a scanning gantry inclination controller 27.

The x-ray generating device 21 and the multi-row x-ray detector 24 turns around the rotation center IC. The vertical direction being supposed to be the y direction, the horizontal direction the x direction and the direction of the table and cradle movement perpendicular to them the z direction, the rotational plane of the x-ray generating device 21 and the multi-row x-ray detector 24 is the xy plane. Further, the moving direction of the cradle 12 is the z direction.

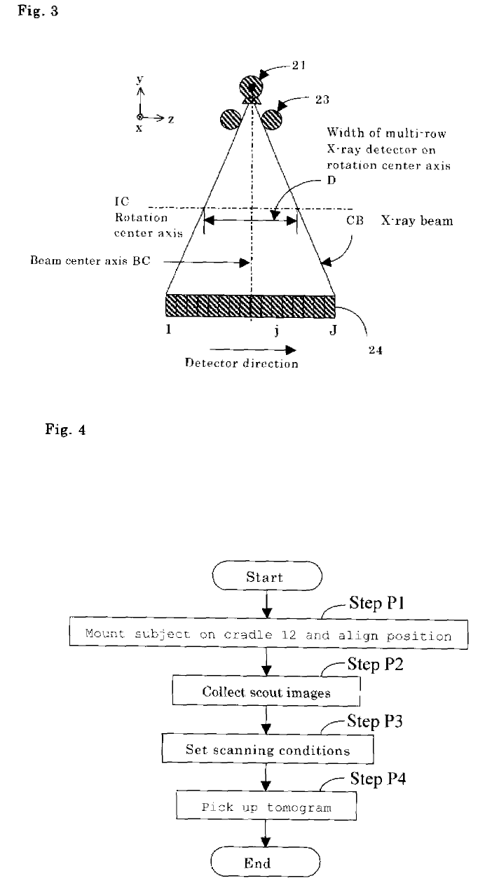

FIG. 2 and FIG. 3 show views of the geometrical arrangement of the x-ray generating device 21 and the multi-row x-ray detector 24 as seen from the xy plane or the yz plane.

The x-ray generating device 21 generates an x-ray beam known as cone beam CB. When the direction of the center axis of the cone beam CB is parallel to the y direction, the view angle is supposed to be 0 degree.

The multi-row x-ray detector 24 has, for instance, 256 detector rows in the z direction. Each x-ray detector row has, for instance, 1024 x-ray detector channels.

As shown in FIG. 2, after an x-ray beam leaving the x-ray focus of the x-ray generating device 21 undergoes such spatial control by the x-ray beam forming filter 28 that more x-rays irradiate the center of the reconstruction area P and less x-rays irradiate the peripheries of the reconstruction area P, x-rays present within the reconstruction area P are absorbed by the subject, and transmitted x-rays are collected by the multi-row x-ray detector 24 as x-ray detector data.

As shown in FIG. 3, the x-ray beam leaving the x-ray focus of the x-ray generating device 21 undergoes control by the x-ray collimator 23 in the slice thickness direction of the tomogram, namely in such a way that the x-ray beam width is D on the rotation center axis IC, and x-rays are absorbed by the subject present near the rotation center axis IC, and transmitted x-rays are collected by the multi-row x-ray detector 24 as x-ray detector data.

Collected projection data following irradiation with x-rays are supplied from the multi-row x-ray detector 24 and subjected to A/D conversion by the DAS 25, and inputted to the data collecting buffer 5 via a slip ring 30. The data inputted to the data collecting buffer 5 are processed by the central processing unit 3 in accordance with a program in the storage unit 7 to be reconstructed into a tomogram, which is displayed on the monitor 6.

FIG. 4 is a flow chart outlining the operation of the x-ray CT apparatus of this embodiment.

At step P1, the subject is mounted on the cradle 12 and aligned. The subject mounted on the cradle 12 undergoes alignment of the reference point of each region to the central position of the slice light of the scanning gantry 20.

At step P2, scout images are collected. Scout images are usually picked up at 0 degree and 90 degree, but in some cases, for instance for the head, only 90-degree scout images are picked up. Details of scout imaging will be described afterwards.

At step P3, scanning conditions are set. Usually, imaging is performed while displaying the position and size of the tomogram to be imaged on the scout image as scanning conditions. In this case, information on the total x-ray dose per round of helical scanning, variable-pitch helical scanning, helical shuttle scanning, conventional scanning (axial scanning) or cine-scanning is displayed. Further in cine-scanning, if the number of revolutions or time length is inputted, x-ray dose information for the number of revolutions or the time length inputted in that interest area will be displayed.

At step P4, tomography is performed. Details of the tomography will be described afterwards.

Two implementation examples of data collection by variable-pitch helical scanning will be described below.

### IMPLEMENTATION EXAMPLE 1

The scanning table 10 or the cradle 12 (hereinafter together referred to as the scanning table 10) is moved in the z direction to collect x-ray data during the acceleration, constant speed operation and deceleration of the scanning table 10, and the operation of the scanning table 10 is completely after the end of collection of x-ray data.

### IMPLEMENTATION EXAMPLE 2

Before the scanning table 10 or the cradle 12 (hereinafter together referred to as the scanning table 10) is moved in the z direction the scanning table 10 is kept at halt; after x-ray data are collected by conventional scanning (axial scanning) or cine-scanning is performed at the fan angle+180 degrees or 360 degrees, or in a plurality of turns, the scanning table 10 is moved to collect x-ray data during the acceleration, constant speed operation and deceleration of the scanning table 10; after the stop of operation of the scanning table 10, conventional scanning (axial scanning) or cine-scanning is performed to collect x-ray data at the fan angle+180 degrees or 360 degrees, or in a plurality of turns while the scanning table 10 is at halt; after that the collection of x-ray data is ended; and irradiation with x-rays is also ended.

### IMPLEMENTATION EXAMPLE 1

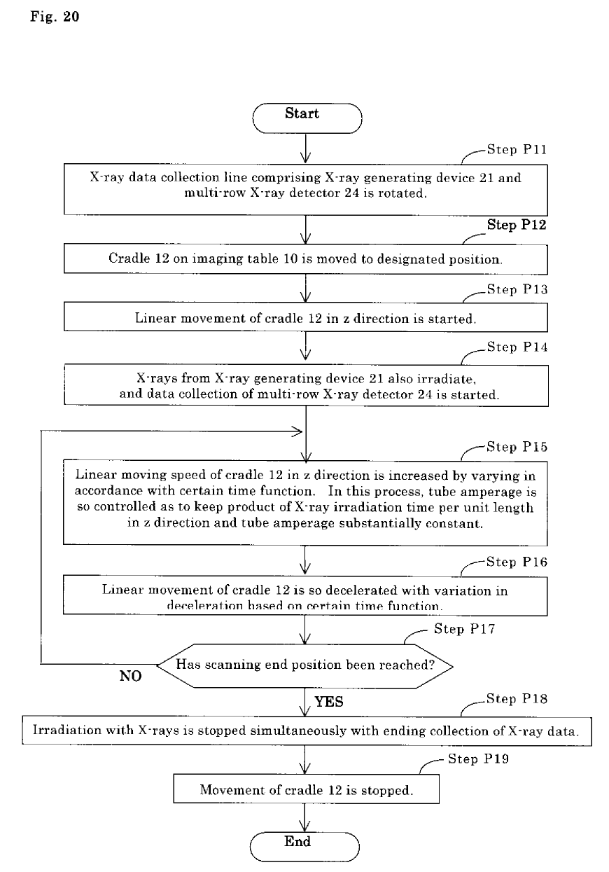

FIG. 20 shows a flow chart of the overall operational flow of this Implementation Example 1.

At step P11, the x-ray data collection line comprising the x-ray generating device 21 and the multi-row x-ray detector 24 is rotated.

At this step, the x-ray data collection line comprising the x-ray generating device 21 and the multi-row x-ray detector 24 may as well be inclined in the z direction from the xy plane.

At step P12, the cradle 12 on the scanning table 10 is moved to a designated position.

In this case, the imaging start position and the imaging end position are set on the user interface screen on the monitor display or the like for setting scanning conditions of tomography in advance. If it is possible to set the imaging start position, the imaging end position and the size of the imaging area on a scout image, it will often contribute to operational ease.

At step P13, the linear movement of the cradle 12 in the z direction is started.

At step P14, x-rays from the x-ray generating device 21 also begin irradiation, and data collection of the multi-row x-ray+detector 24 is started.

If data collection is to be started during the acceleration of the linear movement of the cradle 12 in the z direction, x-ray data are collected while measuring the z-directional coordinate position of each view. Or x-ray data are collected while correctly predicting the z-directional coordinate position.

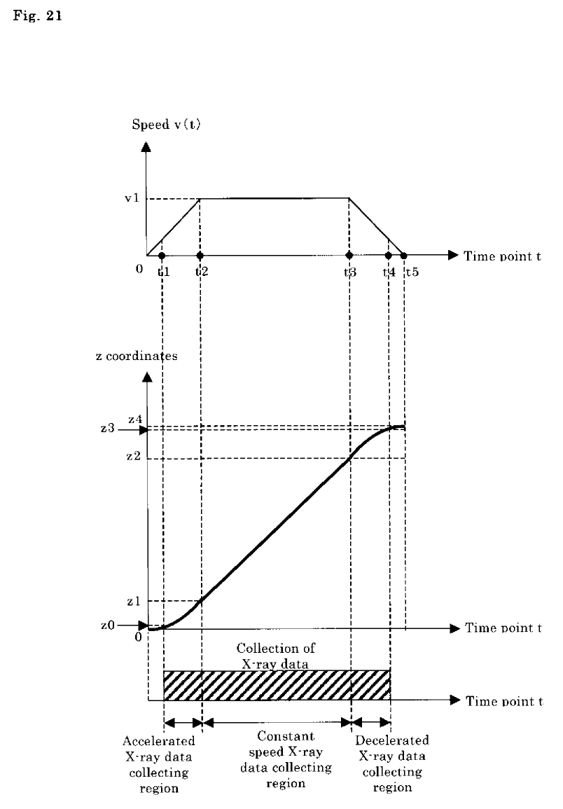

At step P15, the linear moving speed of the cradle 12 in the z direction is increased by varying in accordance with a certain time function. In this process, the tube amperage is so controlled as to keep the product of the x-ray irradiation time per unit length in the z direction and the tube amperage substantially constant. FIG. 21 shows an example of the time function of speed.

Within the accelerating range of the cradle 12, the speed of the cradle is still slow, and the subject may be exposed to a high dose of x-rays. For this reason, if the product of the x-ray irradiation time per unit length in the z direction and the tube amperage is kept constant, the unnecessary exposure of the subject can be reduced.

At step P16, the linear moving speed of the cradle 12 is so decelerated with the variation in deceleration based on a certain time function.

At step P17, it is judged whether or not the scanning end position has been reached and, if YES, the flow will move ahead to step P18 or, if NO, to step P15.

At step P18, irradiation with x-rays is stopped at the same time as ending the collection of x-ray data.

At step P19, the movement of the cradle 12 is stopped.

FIG. 21 illustrates the operation of Implementation Example 1.

The speed v(t) of the scanning table 10 or the cradle 12 accelerates between time points 0 and t2, stays at a constant speed v1 between time points t2 and t3, and decelerates between time points t3 and t5.

As a result of the movement of the scanning table 10 or the cradle 12, if the z-directional coordinate position to be imaged is z=z0 at the time point t0, the imaging position will be z=z0 at the time point t1, z=z1 at the time point t2, z=z2 at the time point t3, z=z3 at the time point t4 and z=z4 at the time point t5.

X-ray data are collected between the time points t1 and t4: between the time points t1 and t2 is an accelerated x-ray data collecting region, between the time points t2 and t3, a constant speed x-ray data collecting region, and between the time points t3 and t4, a decelerated x-ray data collecting region. No x-ray data are collected between the time points 0 and t1 and between t4 and t5.

### IMPLEMENTATION EXAMPLE 2

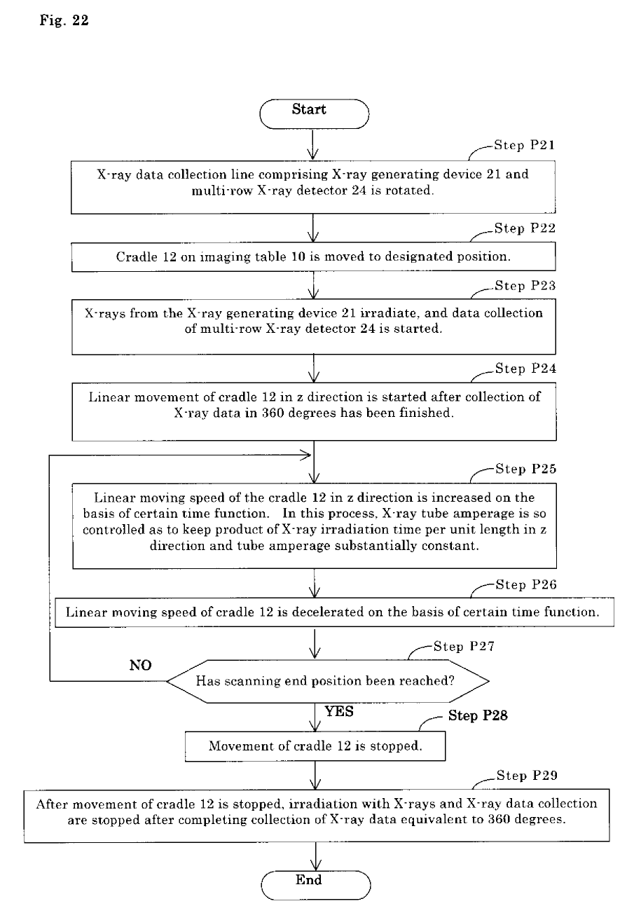

FIG. 22 shows a flow chart of the overall operational flow of Implementation Example 2.

At step P21, the x-ray data collection line comprising the x-ray generating device 21 and the multi-row x-ray detector 24 is rotated. At this step, the x-ray data collection line comprising the x-ray generating device 21 and the multi-row x-ray detector 24 may as well be inclined in the z direction from the xy plane.

At step P22, the cradle 12 on the scanning table 10 is moved to a designated position. In this case, the imaging start position and the imaging end position are set on the user interface screen on the monitor display or the like for setting scanning conditions of tomography in advance. If it is possible to set the imaging start position, the imaging end position and the size of the imaging area on a scout image, it will also contribute to operational ease, too.

At step P23, x-rays from the x-ray generating device 21 begin irradiation, and data collection of the multi-row x-ray detector 24 is started. During x-ray data collection, from the time x-ray data collection line is still at halt, x-ray data collection is performed while measuring the z-directional coordinate position in the x-ray projection data of each view. Alternatively, x-ray data are collected while predicting the directional coordinate position.

At step P24, the linear movement of the cradle 12 in the z direction is started after collection of x-ray data in 360 degrees has been finished.

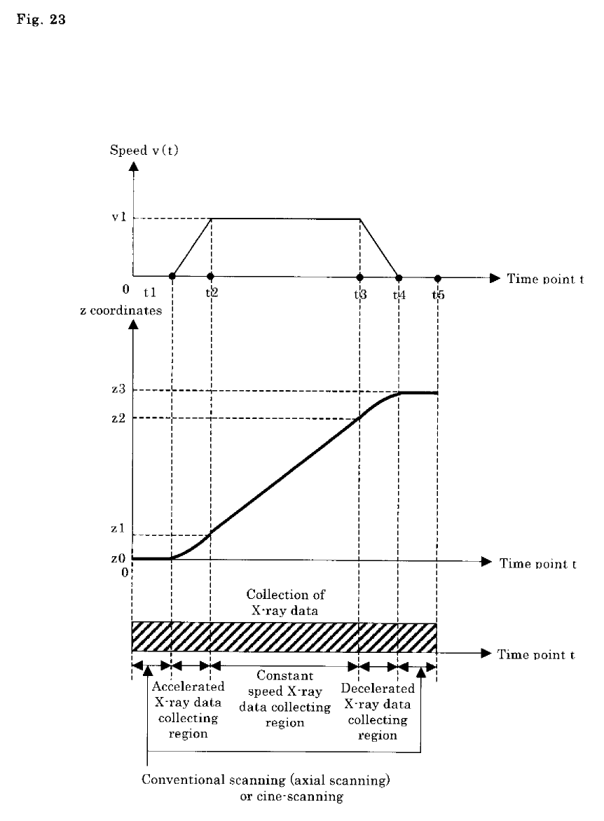

At step P25, the linear moving speed of the cradle 12 in the z direction is increased by varying in accordance with a certain time function. In this process, the x-ray tube amperage is so controlled as to keep the product of the x-ray irradiation time per unit length in the z direction and the tube amperage substantially constant. FIG. 23 shows an example of the time function of speed. Within the accelerating range of the cradle 12, the speed of the cradle is still slow, and the subject may be exposed to a high dose of x-rays. For this reason, if the product of the x-ray irradiation time per unit length in the z direction and the tube amperage is kept constant, the unnecessary exposure of the subject can be reduced.

At step P26, the linear moving speed of the cradle 12 is decelerated on the basis of a certain time function.

At step P27, it is judged whether or not the scanning end position has been reached and, if YES, the flow will move ahead to step P28 or, if NO, to step P25.

At step P28, the movement of the cradle 12 is stopped.

As step S29, after the movement of the cradle 12 is stopped, irradiation with x-rays and x-ray data collection are stopped after completing the collection of x-ray data equivalent to 360 degrees.

FIG. 23 illustrates the operation of Implementation Example 2.

The speed v(t) of the scanning table 10 or the cradle 12 is at halt between the time points 0 and t1, accelerates between the time points t1 and t2, moves at a constant speed v1 between the time points t2 and t3, decelerates between the time points t3 and t4, and is at halt between the time points t4 and t5.

As a result of the movement of the scanning table 10 or the cradle 12, if the z-directional coordinate position to be imaged is z=z0 at the time point t0, the imaging position will be z=z0 between the time points 0 and t1, z=z1 at time point t2, z=z2 at the time point t3, z=z3 between the time points t4 and t5.

X-ray data are collected between time points t1 and t5: between time points t0 and t1 is a region of conventional scanning (axial scanning) or cine-scanning, between the time points t1 and t2 is a region of accelerated x-ray data collection, between the time points t2 and t3 is a constant speed x-ray data collecting region, between the time points t3 and t4 is a region of decelerated x-ray data collection, and, between the time points t4 and t5 is a region of conventional scanning (axial scanning) or cine-scanning.

Data collection of variable-pitch helical scanning is carried out by the x-ray data collection in Implementation Example 1 or Implementation Example 2 described above.

However, though the scanning table 10 or the cradle 12 is moved in Implementation Example 1 and Implementation Example 2, the same effect can be achieved by moving the scanning gantry 20.

Further, though the flow chart of FIG. 22 for Implementation Example 2 supposes 360 degrees for x-ray data collection by conventional scanning (axial scanning) or cine-scanning, the same effect can be achieved by half-scanning at the fan angle+180 degrees or by cine-scanning by more than one turn.

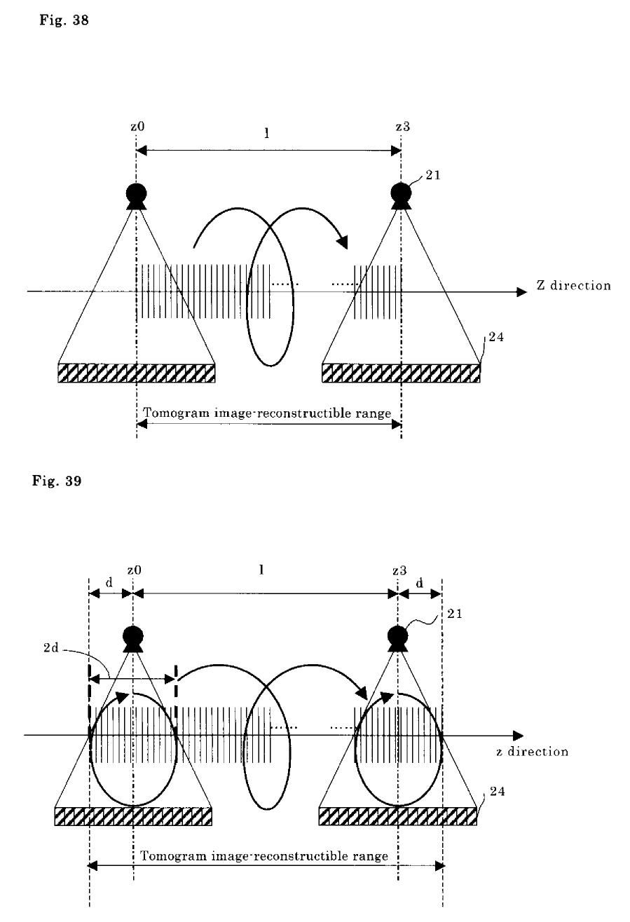

Incidentally, whereas the duration of x-ray data collection in Implementation Example 1 is as shown in FIG. 21, the range in which tomographic images can be reconstructed would conceivably be as shown in FIG. 38. X-ray data are collected between the time points t1 and t4, and the x-ray data collection line moves in this while over a distance of 1=z3−z0 between the z-directional coordinates z0 and z3.

To add, during this period between z0 and z3, the accelerated x-ray data collecting region undergoes variable-pitch helical scanning, the constant speed x-ray data collecting region undergoes helical scanning, and the decelerated x-ray data collecting region undergoes variable-pitch helical scanning. Since every region undergoes helical scanning, tomograms cannot undergo image reconstruction in the range where the z-directional coordinate is smaller than z0 and in the range where the z-directional coordinate is greater than z3. For this reason, the range of tomographic image reconstruction is in the part of distance 1 of [z0, z3].

On the other hand, the duration of x-ray data collection in Implementation Example is such that, as shown in FIG. 23, x-ray data are collected from the time point 0 until the time point t5, and x-ray data collection line moves in this while over a distance of 1=z3−z0 between the z-directional coordinates z0 (where z0=0) and z3.

Incidentally, in this distance between z0 and z3, the accelerated x-ray data collecting region undergoes variable-pitch helical scanning, the constant speed x-ray data collecting region, helical scanning, and the decelerated x-ray data collecting region, variable-pitch helical scanning.

In addition to this, at the points z=z0 and z=z3, conventional scanning (axial scanning) or cine-scanning is further performed. It is now supposed that width of the x-ray beam in the z direction at the rotation center of the x-ray data collection line is 2d. In this case, both in the range where the z-directional coordinate is smaller than z0 [z0−d, z0] and in the range where the z-directional coordinate is greater than z3 [z3, z3 d], tomography is also possible by conventional scanning (axial scanning) or cine-scanning. For this reason, image reconstruction of tomograms in Implementation Example 2 takes in the part of 1+2d in distance to [z0−d, z3+d].

Thus, to compare Implementation Example 1 and Implementation Example 2, while irradiation with x-rays by conventional scanning (axial scanning) or cine-scanning at the points z=z0 and z=z3 are greater by the fan angle+180 degrees or 360 degrees in Implementation Example 2, the range where tomographic image reconstruction is possible is correspondingly increased by d each forward and backward in the z direction or by a total of 2d.

Considering from the viewpoint of the movable range of the scanning table 10 or the cradle 12, while the moving distance of the x-ray data collection line is [z0, z3] both in Implementation Example 1 and in Implementation Example 2, the range where tomographic image reconstruction is possible is increased by d each forward and backward in the z direction or by a total of 2d.

Considering from the viewpoint of image reconstruction, this need in Implementation Example 1 can be addressed only by an image reconstruction algorithm for helical scanning, which is variable-pitch helical scanning in which the moving distance of the scanning table 10 or the cradle 12 per view varies, Implementation Example 2 requires an image reconstruction algorithm for conventional scanning (axial scanning) or cine-scanning, in addition to that for the variable-pitch helical scanning. Therefore, image reconstruction is performed while switching over between these two image reconstruction algorithms in the course of consecutive image reconstruction of tomograms.

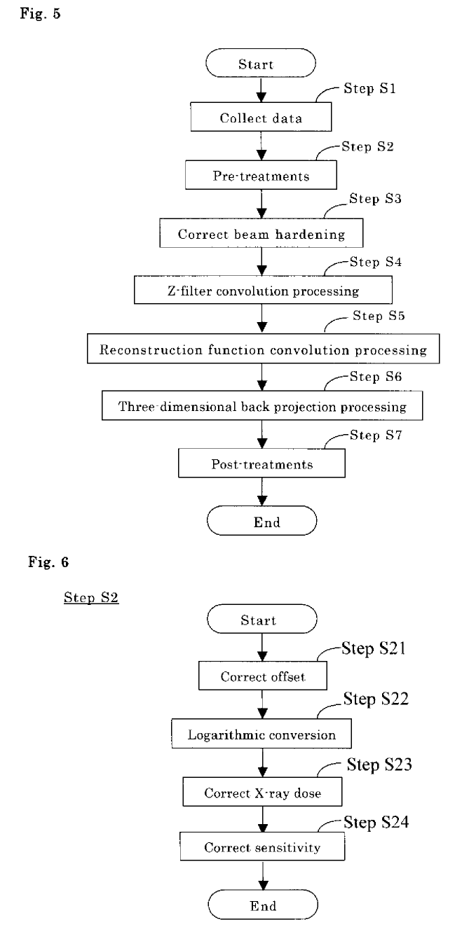

FIG. 5 is a flow chart outlining the operations of tomography and scout imaging by the x-ray CT apparatus 100 according to the invention.

At step S1, in helical scanning, x-ray detector data are collected while rotating the x-raytube 21 and the multi-row x-ray detector 24 around the subject and linearly moving the cradle 12 on the table, the x-ray detector data being collected by adding the z-direction position z table (view) to x-ray detector data DO (view, j, i) represented by the view angle view, the detector row number j and the channel number i. In helical scanning, data area collected in a constant speed range.

In variable-pitch helical scanning or helical shuffle scanning, data collection in helical scanning is performed not only in a constant speed range but also data collection is carried out during acceleration and during deceleration.

Further, in conventional scanning (axial scanning) or cine-scanning, x-ray detector data are collected by rotating the data collection line one round or a plurality of rounds while keeping the cradle 12 on the scanning table 10 fixed in a certain z-directional position. X-ray detector data are further collected by rotating the data collection line one round or a plurality of rounds as required after moving to the next z-directional position.

On the other hand, in scout imaging, x-ray detector data are collected while keeping the x-ray tube 21 and the multi-row x-ray detector 24 fixed and linearly moving the cradle 12 on the scanning table 10.

At step S2, x-ray detector data D0 (view, j, i) are pre-treated to be converted into projection data. The pre-treatments comprise offset correction at step S21, logarithmic conversion at step S22, x-ray dose correction at step S23 and sensitivity correction at step S24 as shown in FIG. 6.

In scout imaging, by displaying the pre-treated x-ray detector data matched with the pixel size in the channel direction and the pixel size in the z direction, which is the linear moving direction of the cradle, matched with the display pixel size of the monitor 6, the scout image is completed.

At step S3, the pre-treated projection data D1 (view, j, i) are subjected to beam hardening correction. The beam hardening correction at step S3 can be expressed in, for instance, a polynomial form as represented below (Mathematical Expression 1), with the projection data having undergone sensitivity correction at S24 of the pre-treatment S2 being represented by D1 (view, j, i) and the data after the beam hardening correction at S3 by D11 (view, j, i).

### [Mathematical Expression 1]

D11(view, j,i)=D1(view, j, i)·(Bo(j,i)+B₁(j, i)·D1(view, j, i)+B₂(j,i)·D₁(view, j,i)²) (Formula 1)

Since each j rows of detectors can be subjected to beam hardening correction independently of others then, if the tube voltage of each data collection line differs from others depending on scanning conditions, differences in detector characteristics from row to row can be compensated for.

At step S4, the projection data D11 (view, j, i) having undergone beam hardening correction are subjected to z filter convolution, by which filtering is done in the z direction (the row direction).

Thus, the data D11 (view, j, i) (i=1 to CH, j=1 to ROW) of the multi-row x-ray detector having undergone beam hardening correction after the pre-treatment at each view angle and on each data collection line are subjected to, for instance, filtering whose row-directional filter size is five rows as represented by (Formula 2) and (Formula 3) below.

$$\begin{matrix}[ Mathematical Expression 2 ] & \\ ( {w}_{1} ( i ) , {w}_{2} ( i ) , {w}_{3} ( i ) , {w}_{4} ( i ) , {w}_{5} ( i ) ) , & ( Formula 2 )\end{matrix}$$

The corrected detector data D12(view, j, i) will be as represented by (Formula 4) below.

$$\begin{matrix}[ Mathematical Expression 3 ] & \\ D 12 ( view , j , i ) = {\sum }_{k = 1}^{5} ( D 11 ( view , j + k - 3 , i ) \cdot {w}_{k} ( j ) ) & ( Formula 4 )\end{matrix}$$

Incidentally, the maximum channel width being supposed to be CH and the maximum row value being ROW, the following (Formula 5) and (Formula 6) will hold.

D11(view,−1,i)=D11(view,0,i)=D11(view1,i) (Formula 5)

D11(view, ROW, i)=D11(view, ROW+1, i)=D11(view, ROW+2, i) (Formula 6)

On the other hand, the slice thickness can be controlled according to the distance from the center of image reconstruction by varying the row-directional filter coefficient from channel to channel. Since the slice thickness is usually greater in the peripheries than at the center of reconstruction in a tomogram, the slice thickness can be made substantially uniform whether in the peripheries or at the center of image reconstruction by so differentiating the row-directional filter coefficient between the central part and the peripheries that the range of the row-direction filter coefficient is varied more greatly in the vicinities of the central channel and varied more narrowly in the vicinities of the peripheral channel.

By controlling the row-directional filter coefficient between the central channels and the peripheral channels of the multi-row x-ray detector 24 in this way, the control of the slice thickness can also be differentiated between the central part and the peripheries. By slightly increasing the slice thickness with the row-directional filter, both artifacts and noise can be substantially improved. The extent of improvement of artifacts and that of noise can be thereby controlled. In other words, a tomogram having undergone three-dimensional image reconstruction, namely picture quality in the xy plane, can be controlled. Another possible embodiment, a tomogram of a thin slice thickness can be realized by using deconvolution filtering for the row-directional (z-directional) filter coefficient.

At step S5, convolution of the reconstructive function is performed. Thus, the result of Fourier transform is multiplied by the reconstructive function to achieve inverse Fourier transform. In the convolution of reconstructive function at S5, data after the z filter convolution being represented by D12, data after the convolution of reconstructive function by D13 and the reconstructive function to be convoluted by Kernel (j), the processing to convolute the reconstructive function can be expressed in the following way.

### [Mathematical Expression 5]

D13(view, j,i)=D12(view, j,i)*Kernel (j) (Formula 7)

Thus, since the reconstructive function Kernel (j) permits independent convolution of the reconstructive function on each j rows of detectors, differences in noise characteristics and resolution characteristics from one row to another can be compensated for.

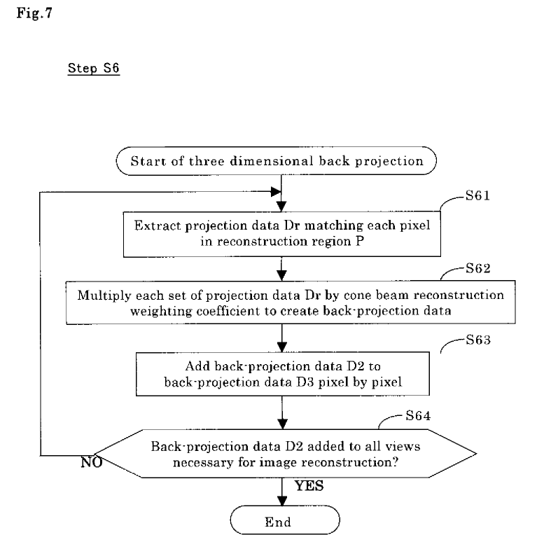

At step S6, the projection data D13 (view, j, i) having undergone convolution of the reconstructive function are subjected to three-dimensional back-projection to obtain back-projected data D3 (x, y, z). The image to be reconstructed is reconstructed into a three-dimensional image on a plane perpendicular to the z axis, the xy plane. The following reconstruction area P is supposed to be parallel to the xy plane. This three-dimensional back-projection will be described afterwards with reference to FIG. 7.

At step S7, the back-projected data D3 (x, y, z) are subjected to image space z-directional filter convolution. The tomogram having undergone the image space z-directional filter convolution being represented by D4 (x, y, z), the following will hold.

$$\begin{matrix}[ Mathematical Expression 6 ] & \\ D 4 ( x , y , z ) = {\sum }_{i = - 1}^{1} D 3 ( x , y , z + i ) \cdot v ( i ) & ( Formula 8 )\end{matrix}$$

In the foregoing, v(i) represents image space z-directional filter convolution coefficients with a width in z direction being 2l+1, which constitute the following sequence of coefficients.

### [Mathematical Expression 7]

v(−l), v(−l+1), . . . v(−1), v(0), v(1), . . . v(l−1), v(l) (Formula 9)

In helical scanning, the image space filter coefficient v(i) may be an image space z-directional filter coefficient not dependent on the z-directional position. However, especially in conventional scanning (axial scanning) or cine-scanning where a two-dimensional x-ray area detector 24 or a multi-row x-ray detector 24 having a large detector width in the z direction, if the image space filter coefficient v(i) is an image space z-directional filter coefficient dependent on the position of the row of x-ray detections in the z direction, it will be even more effective because it makes possible detailed adjustment dependent on the row position of each tomogram.

At step S8, a tomogram D4 (x, y, z) having undergone image space z-directional filter convolution is subjected to post-treatments including image filter convolution and CT value conversion to obtain a tomogram D41 (x, y).

In the image filter convolution as post-treatment, with the data having gone through three-dimensional back-projection being represented by D41 (x, y, z), the data having gone through image filter convolution by D42 (x, y, z) and the image filter by Filter (z):

### [Mathematical Expression 8]

D42(x, y, z)=D41(x, y, z)*Filter(z) (Formula 10)

Thus, as independent mage filter convolution can be processed for each j rows of detectors, differences in noise characteristics and resolution characteristics from one row to another can be compensated for.

The obtained tomogram is displayed on the monitor 6.

FIG. 7 is a flow chart showing details of the three-dimensional back-projection processing, step S6 in FIG. 5.

In this embodiment, the image to be reconstructed is reconstructed into a three-dimensional image on a plane perpendicular to the z axis and the xy plane. The following reconstruction area P is supposed to be parallel to the xy plane.

At step S61, note is taken on one view out of all the views needed for image reconstruction of a tomogram (namely 360-degree views or “180-degree+fan angle” views), and projection data Dr corresponding to the pixels in the reconstruction area P are extracted.

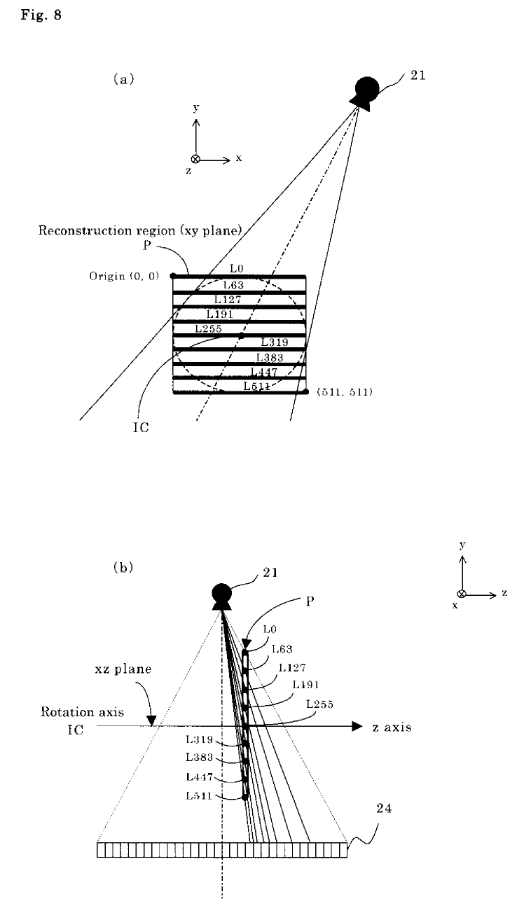

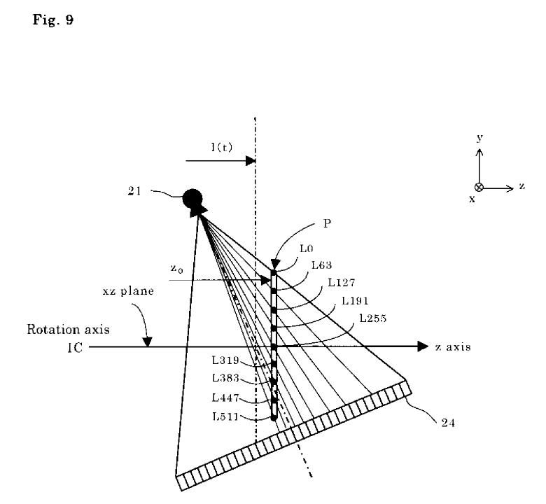

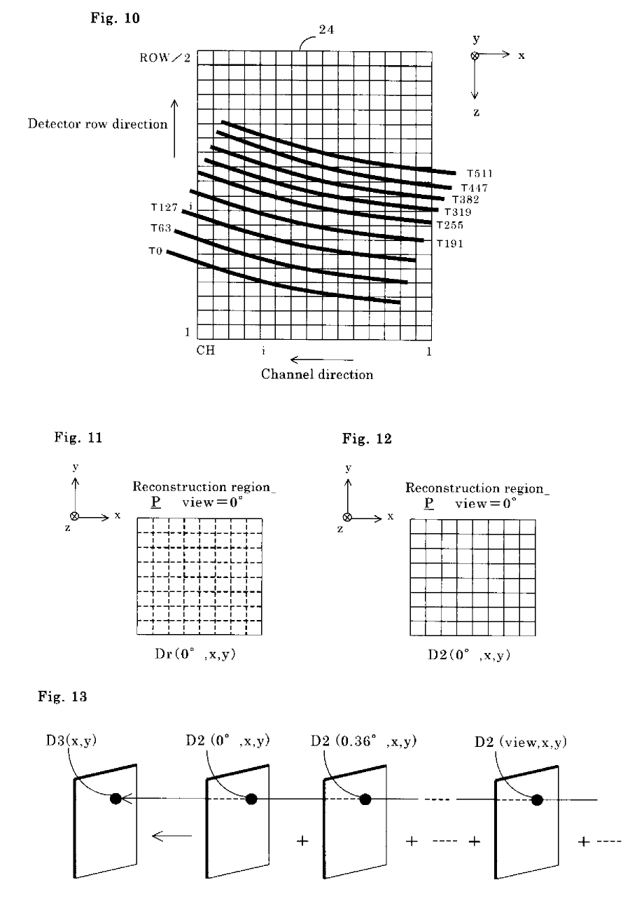

As shown in FIG. 8(a) and FIG. 8(b), a square area of 512×512 pixels parallel to the xy plane being supposed to be the reconstruction area P, and a pixel row L0 of y=0, a pixel row L63 of y=63, a pixel row L 127 of y=127, a pixel row L191 of y=191, a pixel row L 255 of y=255, a pixel row L319 of y=319, a pixel row L383 of y=383, a pixel row L447 of y=447 and a pixel row L511 of y=511, all parallel to the x-axis of y=0, being taken as rows, if projection data on lines T0 through T511 are extracted as shown in FIG. 10, wherein these pixel rows L0 through L511 are projected on the plane of the multi-row x-ray detector 24 in the x-ray transmitting direction, they will constitute projection data Dr (view, x, y) of pixel rows L0 through L511. It is provided, however, that x and y match pixels (x, y) in the tomogram. A case in which the data collection line is inclined is shown in FIG. 9.

The x-ray transmitting direction is determined by the geometrical positions of the x-ray focus of the x-ray tube 21, the pixels and the multi-row x-ray detector 24. However, since the z coordinate z (view) of the x-ray detector data D0 (view, j, i) is known as the z direction of the linear table movement Z table (view) attached to the x-ray detector data, the x-ray transmitting direction can be accurately figured out in the data collection geometric system of the x-ray focus and the multi-row x-ray detector even if the x-ray detector data D0 (view, j, i) are obtained during acceleration or deceleration.

Incidentally, if part of the lines goes out of the channel direction of the multi-row x-ray detector 24 as does, for instance, the line T0 resulting from the projection of the pixel row L0 onto the plane in the multi-row x-ray detector 24 in the x-ray transmitting direction, the matching projection data Dr (view, x, y) are set to “0”. Or if they go out of the z direction, it will be figured out by extrapolating projection data Dr (view, x, y).

In this way, projection data Dr (view, x, y) matching the pixels of the reconstruction area P can be extracted as shown in FIG. 11.

Referring back to FIG. 7, at step S62, projection data Dr (view, x, y) are multiplied by a cone beam reconstruction weighting coefficient to create projection data D2 (view, x, y) shown in FIG. 12.

The cone beam reconstruction weighting coefficient w (i, j) here is as follows. In reconstructing a fan beam image, the following relationship generally holds (Formula 9) where y is the angle which a straight line linking the focus of the x-ray tube 21 and a pixel g (x, y) on the reconstruction region P (on the xy plane) forms with respect to the center axis Bc of the x-ray beam where view=βa and the view opposite thereto is view=βb:

### [Mathematical Expression 9]

βb=βa+180°−2γ (Formula 9)

With the angles fonned by the x-ray beam passing the pixel g (x, y) on the reconstruction region P and the x-ray beam opposite thereto with respect to the reconstruction plane P being respectively represented by αa and αb, the back-projected pixel data D2 (0, x, y) are figured out by adding after multiplication with reconstruction weighting coefficients ωa and ωb. In this case, (Formula 10) holds.

### [Mathematical Expression 10]

D2(0,x,y)=ωa·D2(0,x,y)_{—}a+ωb·D2(0,x,y)_{—}b (Formula 10)

where D2 (0, x, y)_a are supposed to be the back-projected data of view βa and D2 (0, x, y)_b, the back-projected data of view βb.

Incidentally, the sum of the mutually opposite beams of cone beam reconstruction weighting coefficients is represented by (Formula 11):

### [Mathematical Expression 11]

ωa+ωb=1 (Formula 11)

By adding the products of multiplication by cone beam reconstruction weighting coefficients ωa and ωb, cone angle artifacts can be reduced.

For instance, reconstruction weighting coefficients ωa and ωb obtained by the following formulas can be used. In these formulas, ga is the weighting coefficient of the view βa and gb, the weighting coefficient of the view βb.

Where ½ of the fan beam angle is γmax, (Formula 12) through (Formula 17) below hold.

### [Mathematical Expression 12]

gb=f(γ max, αa, βa) (Formula 12)

gb=f(γ max, αb, βb) (Formula 13)

xa=2·ga^{q}/(ga^{q}+gb^{q}) (Formula 14)

xb=2·gb^{q}/(ga^{q}+gb^{q}) (Formula 15)

wa=xa²·(3−2xa) (Formula 16)

wb=xb²·(3−2xb) (Formula 17)

### (For instance, q=1 is supposed.)

For instance, if max[ ] is supposed to be a function taking up what is greater in value as an example of ga and gb, (Formula 18) and (Formula 19) below will hold.

### [Mathematical Expression 13]

ga=max[0,{(π/2+γ max)−|βa|}]·|tan(αa)| (Formula 18)

gb=max[0,{(π/2+γ max)−|βb|}]·|tan(αb)| (Formula 19)

In the case of fan beam image reconstruction, each pixel of the reconstruction region P is firther multiplied by a distance coefficient. The distance coefficient is (r1/r0)2 where r0 is the distance from the focus of the x-ray tube 21 to the detector row j and the channel i of the multi-row x-ray detector 24 matching the projection data Dr, and r1 is the distance from the focus of the x-ray tube 21 to a pixel matching the projection data Dr on the reconstruction region P.

In the case of parallel beam image reconstruction, it is sufficient to multiply each pixel of the reconstruction region P only by the cone beam reconstruction weighting coefficient w (i, j).

At step S63, projection data D2 (view, x, y) are added, correspondingly to pixels, to back-projected data D3 (x, y) cleared in advance as shown in FIG. 13.

At step S64, steps 61 through S63 are repeated for all the views necessary for CT image reconstruction (namely 360-degree views or “180-degree+fan angle” views) to obtain back-projected data D3 (x, y) as shown in FIG. 13.

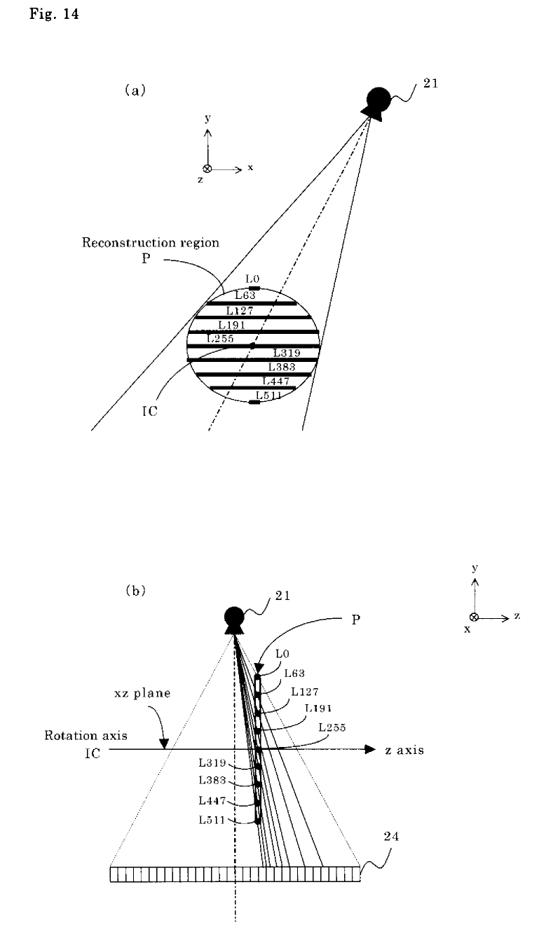

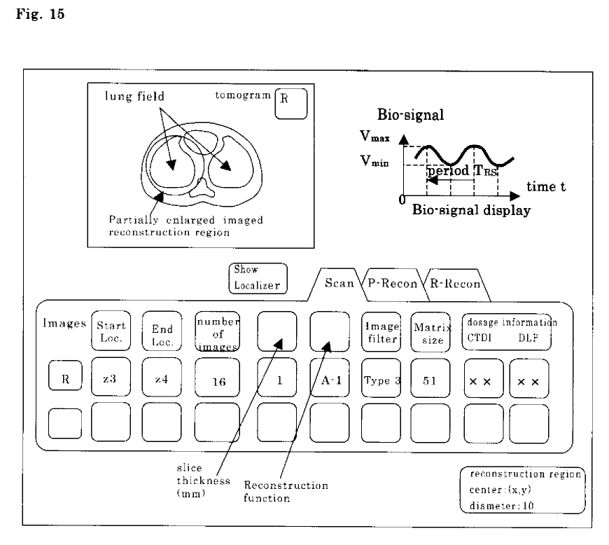

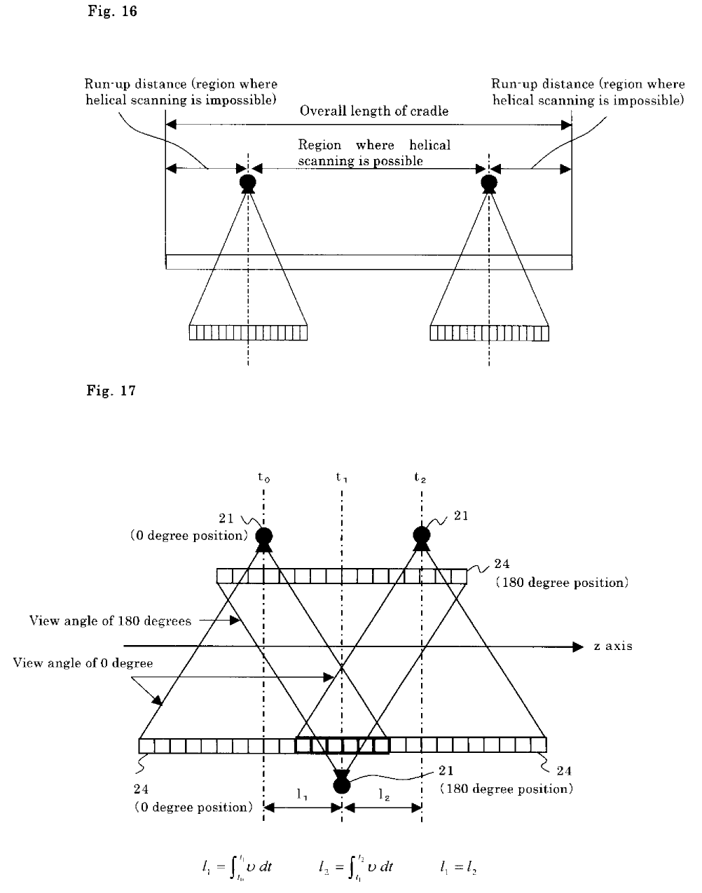

Incidentally, the reconstruction region P may as well be a circular area of 512 pixels in diameter as shown in FIG. 14(a) and FIG. 14(b) instead of a square area of 512×512 pixels.

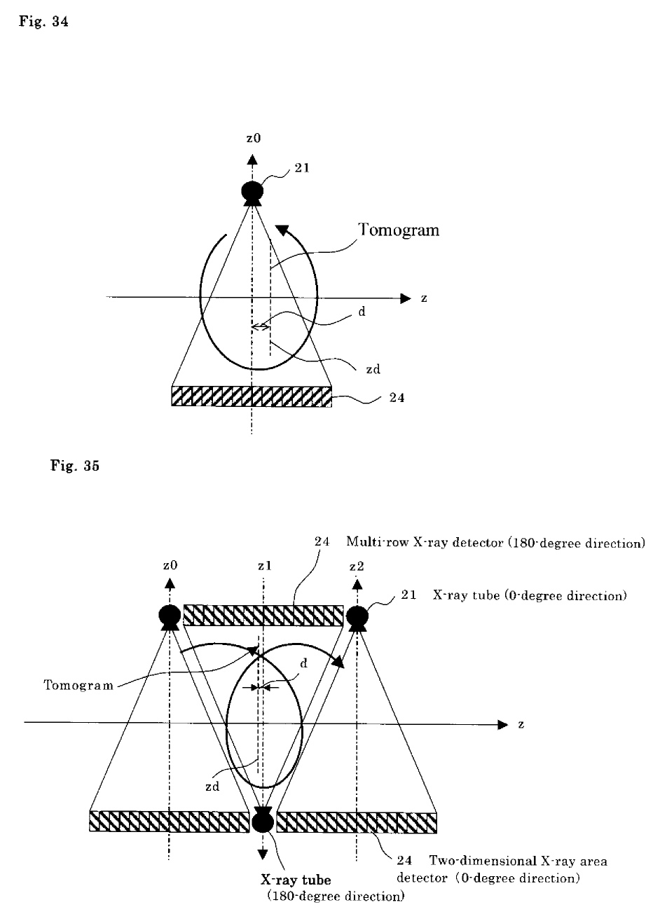

Since the positional relationship between the z-coordinate position z0 of the data collection line and the z-coordinate position zd of the tomogram is constant all the time in conventional scanning (axial scanning) or cine-scanning as shown in FIG. 34, three-dimensional back projection can be processed by multiplication by only this weighting coefficient for cone beam reconstruction in conventional scanning (axial scanning) or cine-scanning.

By contrast, since the positional relationship between the z-coordinate positions z0, z1 and z2 of the data collection line and the z-coordinate position zd of the tomogram varies constantly in helical scanning or variable-pitch helical scanning as shown in FIG. 35, a weighting coefficient hw(d) dependent on the distance d between the data collection line and the tomogram in each of these views, or a weighting coefficient hw (view) for predicting the distance d to the tomogram from each view to figure out the weighting coefficient, is required in addition to this weighting coefficient for cone beam reconstruction in helical scanning or variable-pitch helical scanning.

In helical scanning, multiplication by this weighting coefficient hw (d) or hw(view) is needed in addition to the weighting coefficient for cone beam reconstruction.

For this reason, especially where conventional scanning (axial scanning) or cine-scanning is followed by acceleration to perform helical scanning, and further followed by deceleration to perform conventional scanning (axial scanning) or cine-scanning finally as in Implementation Example 2, it is necessary to make possible in advance the use of two image reconstruction algorithms including the image reconstruction algorithm for conventional scanning (axial scanning) or cine-scanning and an image reconstruction algorithm for helical scanning.

In this case, there may as well be made ready two image reconstruction algorithms including an image reconstruction algorithm for conventional scanning (axial scanning) or cine-scanning without a weighting coefficient hw (d) or hw(view) and an image reconstruction algorithm for helical scanning having a weighting coefficient hw(d) or hw(view).

Alternatively, in the case of helical scanning for which the weighting coefficient hw (d) or the weighting coefficient hw(view) is provided with a parameter, it may be so arranged that a coefficient dependent on the positional relationship between the data collection line and the tomogram and another coefficient dependent on the distance between the data collection line and the tomogram are outputted, the output being a fixed value or “1” in the case of conventional scanning (axial scanning) or cine-scanning, and switching-over between the two image reconstruction algorithms including the image reconstruction algorithm for conventional scanning (axial scanning) or cine-scanning and the image reconstruction algorithm for helical scanning is made possible according to the parameter.

Incidentally, to consider the relationship between each view angle and the z-directional coordinate position, the following will hold in helical scanning in the constant speed region or normal helical scanning.

As shown in FIG. 17, in one round of helical scanning, there is an advance by a view angle of 0 degree at the time point t0, a view angle of 180 degrees at the time point t1 and a view angle of 0 degree at the time point t2 or in terms of distance in the z direction l1 between the time points t0 and t1 and l2 between the time points t1 and t2. The table speed being constant in this process, l1 and l2 will be represented by (Formula 20), (Formula 21) and (Formula 22) below.

### [Mathematical Expression 14]

l₁=∫ₜ_{₀}^{t}^{₁}v(t)dt (Formula 20)

l₂=∫ₜ_{₁}^{t}^{₂}v(t)dt (Formula 21)

l₁=l₂ (Formula 22)

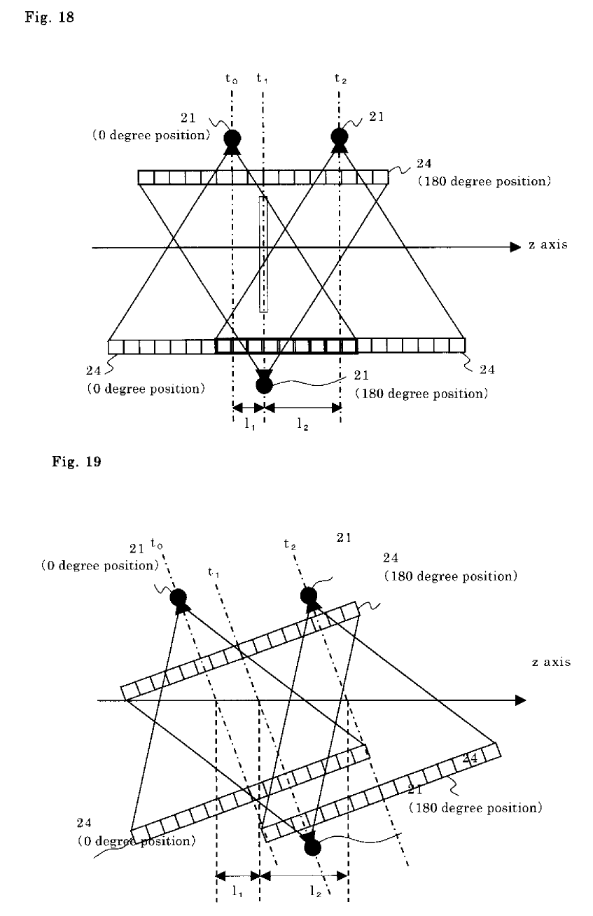

Thus, the view angle and the z-directional coordinate position are in a proportional and linear relationship. However, in variable-pitch helical scanning, the following will hold. Further, the case of variable-pitch helical scanning will be shown next in FIG. 18.

FIG. 19 shows the case of variable-pitch helical scanning where the data collection line is inclined. Assuming one round of helical scanning in every instance, the view angle is 0 degree at the time point t0, the view angle is 180 degrees at the time point t1 and the view angle is 0 degree at the time point t2.

With the distances l1 and l2 advanced in the z direction at a table speed of v(t) then are represented by (Formula 23) and (Formula 24) below.

### [Mathematical Expression 15]

l₁=∫ₜ_{₀}^{t}^{₁}v(t)dt (Formula 23)

l₂=∫ₜ_{₁}^{t}^{₂}v(t)dt (Formula 24)

In this case, l₁ and l₂ are not always equal. This enables the position of the data collection line in the z direction to be measured or predicted. The position l(t) of the data collection line in the z direction at the point of time 1 can be represented by (Formula 25) below.

### [Mathematical Expression 16]

l(t)=∫ₜ_{₀}^{t}^{₁}v(t)dt (Formula 26)

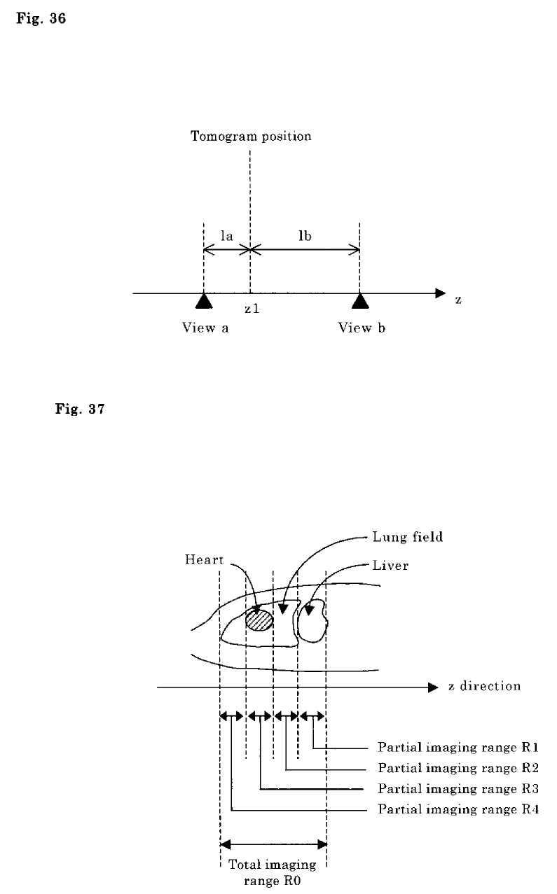

Thus, the view angle and the z-directional coordinate position are not in a proportional or linear relationship. However, if there are an image reconstructing position z1, a certain view a and another view b opposite to it as shown in FIG. 36, a method of multiplying the view a by a weighting coefficient of (Formula 26) and the view b by a weighting coefficient of (Formula 27) is conceivable as an example of the use of weighting coefficients,

### [Mathematical Expression 17]

la/(la+lb) (Formula 26)

lb/(la+bb) (Formula 27)

Alternatively, multiplying by weighting coefficients having (Formula 26) and (Formula 27) as parameters could achieve the same purpose.

By multiplying each set of view data by a weighting coefficient, image reconstruction by variable-pitch helical scanning can be achieved.

As described above, the slice thickness can be controlled by using at least one or combining some of the following methods for image reconstruction

1. z filter convolution.

2. Image reconstruction by multiplying each view of x-ray projection data by a weighting coefficient.

3. Weighted addition processing of images resulting from the multiplication by weighting coefficients of image-reconstructed tomograms consecutive in the z direction.

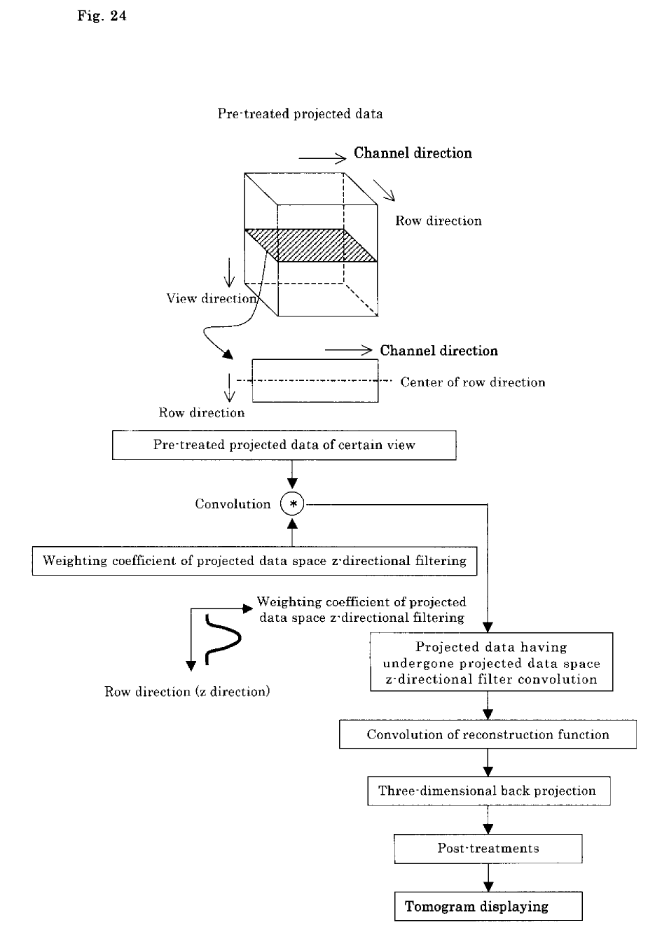

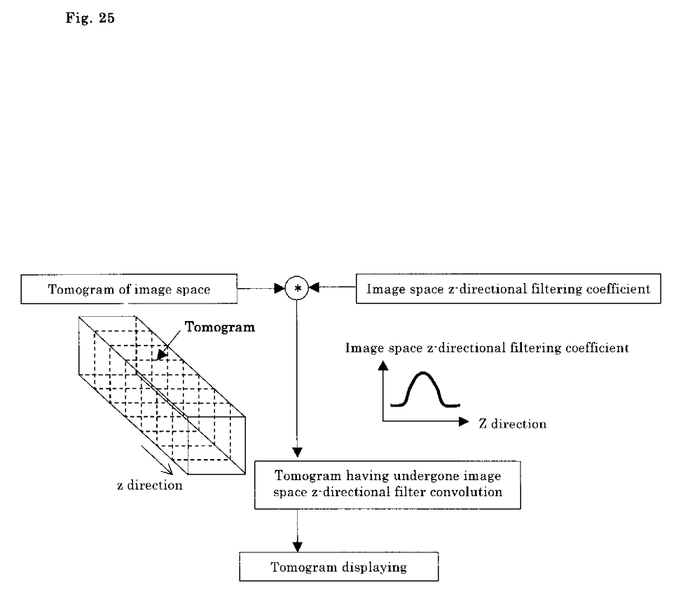

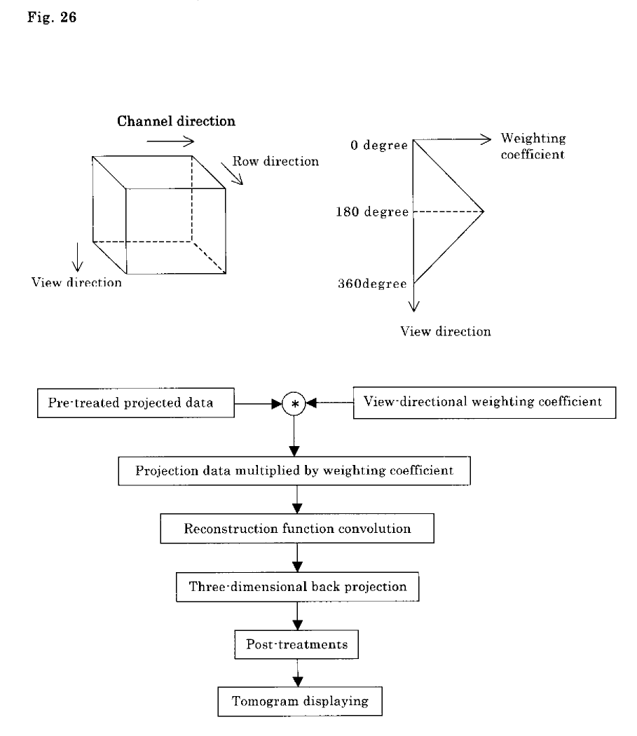

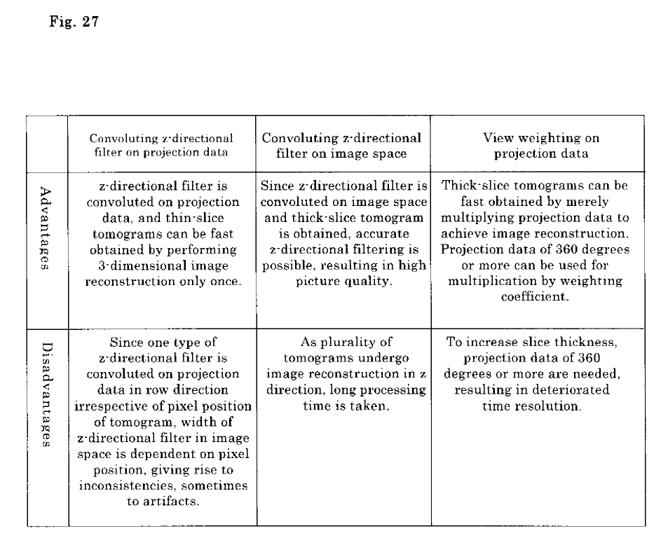

Generally, as stated in the table of FIG. 27, techniques for controlling the slice thickness in x-ray CT apparatuses include the method of z-directional filter convolution on projection data shown in FIG. 24, the method of z-directional filter convolution on image space data shown in FIG. 25, and the method of weighted view processing on projection data shown in FIG. 26.

As stated in the table of FIG. 27, the advantages of the method of z-directional filter convolution on projection data include the availability of tomograms having a large slice thickness by convoluting the z-directional filter on the projection data and performing three-dimensional image reconstruction only once. The disadvantages the method of z-directional filter convolution on projection data include the dependence of the width of the z-directional filter in the image space on the position of each pixel, because one type of z-directional filter is convoluted on the projection data in the row direction irrespective of the positions of pixels in the tomogram, resulting in inconsistencies in the width of the back-projected x-ray beam and accordingly the occurrence of artifacts.

On the other hand, the advantages of the method of z-directional filter convolution on image space include accurate z-directional filter processing and the resultant high picture quality of the tomograms because tomograms having a large slice thickness can be obtained by convoluting the z-directional filter on image space. The disadvantages of the method of z-directional filter convolution on image space include a long processing time taken because a plurality of tomograms are image-reconstructed in the z direction.

The advantages of the method of weighted view processing on projection data views include the fast availability of tomograms having a large slice thickness by mere multiplication by weighting coefficients on the projection data to achieve image reconstruction. Another advantage is that multiplication of projection data of 360 degrees or more by weighting coefficients is possible. The disadvantages ofthe method of weighted view processing on projection data view include a deterioration in time-resolution because obtaining a large slice thickness requires projection data of 360 degrees or more.

Thus, each of these three techniques for controlling the slice thickness has its own advantages and disadvantages. In smaller multi-row x-ray detectors of only about 16 rows even for a multi-row x-ray detector 24 and an x-ray detector width of about 20 mm in the z direction, the method of z-directional filter convolution on projection data has been in general use in conventional practice. The reason is that, since image back projection conventionally takes a long time, and the z-directional filter convolution on projection data space, which needs less frequent image back projection has been preferred over the z-directional filter convolution on image space which requires much more frequent image back projection.

In the z-directional filter convolution on projection data space, a weighting coefficient filter is convoluted in the z direction, which is the row direction, on the projection data and after that the convolution of reconstructing function and image back projection are required only once each, taking only a short time to reconstruct an image.

However, as the x-ray detector width of the multi-row x-ray detector 24 in the z direction has increased, inconsistencies have come to occur sometimes in the z-directional filter convolution on projection data. For instance, it is supposed that the slice thickness of the tomogram to be sought at the center of reconstruction projected on the x-ray detector is equivalent to four times the width of the z-directional filter as shown in FIG. 10. In this case, in three-dimensional image reconstruction, projection data convoluted by the z-directional filter of the width equivalent to four rows is back-projected three-dimensionally irrespective of the positions of pixels in the tomogram.

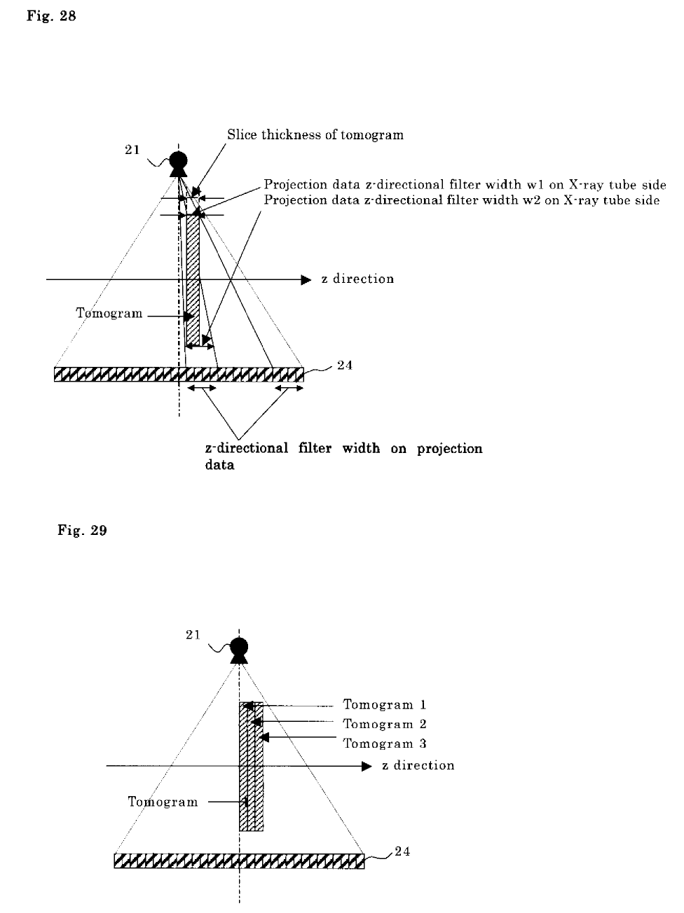

However, as shown in FIG. 28, the width of the projection data z-directional filter in the pixels of the tomogram on the x-ray tube 21 side is w1. The width of the projection data z-directional filter on the multi-row x-ray detector 24 side is w2. In this case, obviously w2>w1.

The greater the slice thickness of the image-reconstructed tomogram, the more significant this phenomenon. Moreover, where the x-ray beam width differs with the position in the tomogram, such as w2>w1, artifacts will occur in the tomogram. Thus, a greater slice thickness of the image-reconstructed tomogram makes it more likely for artifacts to arise in projection data z-directional filter convolution.

In helical scanning, the higher the helical pitch, the greater the difference in the z-directional position of the data of x-ray beam widths w1 and w2, making it even easier for artifacts to arise.

On the other hand, in the z-directional filter convolution of image space, tomograms 1, 2 and 3 of a smaller slice thickness are subjected to image reconstruction in advance as shown in FIG. 29. In this instance, the tomograms of a smaller slice thickness are less subject to inconsistencies due to differences in x-ray beam width with the positions of pixels in tomograms with the result that artifacts are less likely to occur and the picture quality is higher. Since the z-directional filter convolution of image space is applied to these images of the smaller slice thickness, which are higher in picture quality, the picture quality of the tomograms of the greater slice thickness which are subjected finally to image reconstruction is also high.

As is evident from the foregoing, projection data space z-directional filter convolution is more suitable for image reconstruction where the slice thickness is smaller, while image space z-directional filter convolution is more suitable for image reconstruction where the slice thickness is greater.

Further to shorten the time taken to accomplish image reconstruction, for image reconstruction where the slice thickness is greater, it is advisable to use projection data space z-directional filter convolution to the maximum slice width not susceptible to artifacts ensuing from inconsistencies in x-ray beam width due to projection data space z-directional filter convolution and, if the slice thickness is to be further increased, to use image space z-directional filter convolution.

To describe it with reference to the flow chart of FIG. 5, a projection data space z-directional filter is convoluted to the maximum slice width not susceptible to artifacts in the projection data space z-directional filter convolution of step S4 and, if the slice thickness needs to be further increased, image reconstruction is performed to the final slice thickness in the image space z-directional filter convolution at step S7. This enables the slice thickness to be controlled by image space z-directional filter convolution.

The balance between the projection data space z-directional filter convolution and the image space z-directional filter convolution in this case depends on the slice thickness and the width of each row of x-ray detector channel in the multi-row x-ray detector 24 in the row direction. It also depends on the helical pitch in helical scanning. In other words, it is advisable to optimally determine the projection data space z-directional filter coefficient and the image space z-directional filter coefficient after these slice thickness, x-ray detector width in the row direction and helical pitch are selected.

Whereas projection data view weighting is a technique from the helical scanning by an x-ray CT apparatus having only one x-ray detector row upward, it is equally effective for two-dimensional x-ray area detectors. While projection data of 360 degrees are normally used in helical scanning, by using projection data on about 10% or 20% more views for image reconstruction, effects of improving the SN ratio and reducing artifacts can be achieved. Further, by adjusting the weighting coefficient to be applied then, the slice thickness can also be controlled. In variable-pitch helical scanning as well, the slice thickness can be controlled by such projection data view weighting for one turn or more.

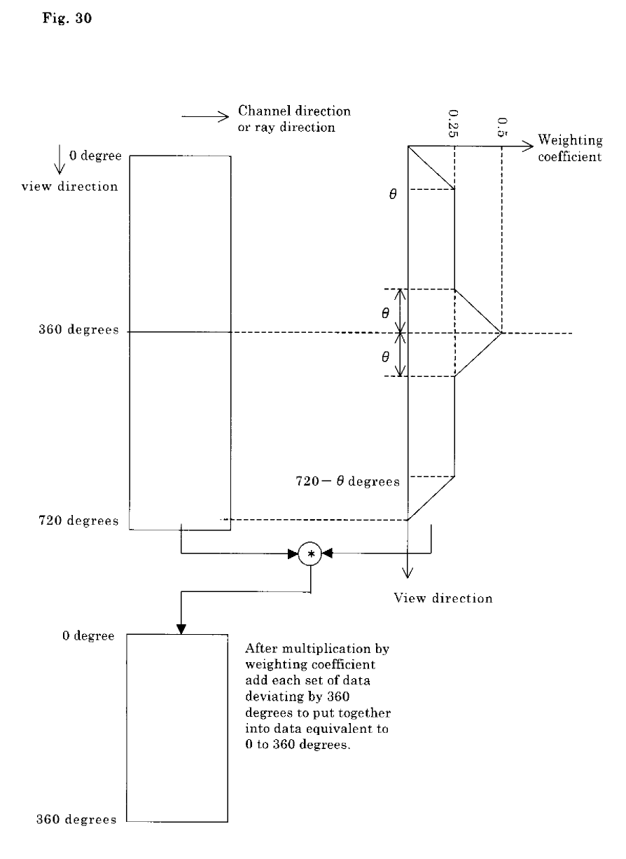

FIG. 30 shows one example of this aspect.

FIG. 30 illustrates projection data after fan-to-parallel conversion has been done. After applying a weighting function in the view direction to projection data expanding in the channel direction or the ray direction and the view direction, they are subjected to reconstructive function convolution, three-dimensional back projection and post-treatments as shown in FIG. 26, and then the tomogram can be displayed. The weighting function in FIG. 30 may be such that the sum of opposite views and views in the same direction becomes 1.0.

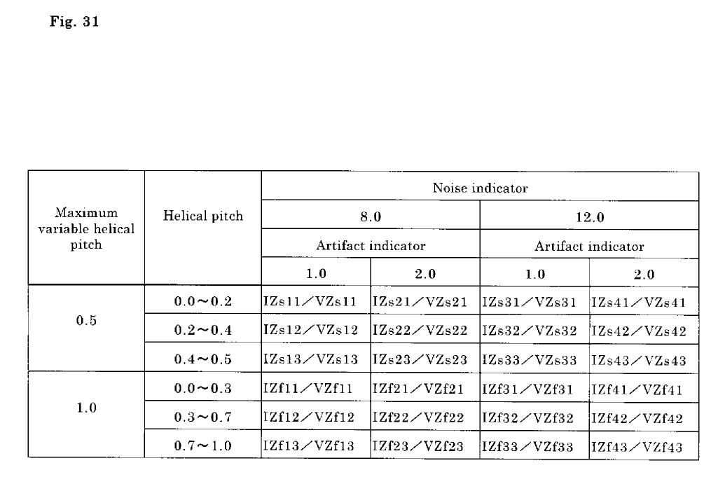

Further, FIG. 31 is a table of projection data space z filter coefficients and image space z filter coefficients under set scanning conditions in variable-pitch helical scanning. By using three-dimensional image reconstruction, tomograms of uniform quality in terms of image noise in the z direction can be obtained even in variable-pitch helical scanning together with x-ray tube current control in the z direction. In other words, tomograms uniform in picture quality characteristics including relative freedom from artifacts, slice thickness and noise in the z direction can be obtained. In this case, it is desirable to optimize the projection data space z filter and image space z filter for each of the differing helical pitches.

In the case of FIG. 31, optimization of the projection data space z filter coefficient and the image space z filter coefficient is carried out with a view to optimizing such picture quality characteristics as the maximum helical pitch noise and artifacts in variable-pitch helical scanning or shuttle mode variable-pitch helical scanning. In this case, besides prescribing the filter coefficient of each at the maximum helical pitch, the projection data space z filter coefficient and the image space z filter coefficient are prescribed to be optimal for each helical pitch because the helical pitch varies from 0 to its maximum. Alternatively, the projection data space z filter coefficient and the image space z filter coefficient may as well be prescribed as functions having the helical pitches as their parameters.

The noise indicators and the artifact indicators in FIG. 31 are targets for picture quality set by scanning condition setting device, which is an scanning condition input screen shown in FIG. 15 for instance. In particular the artifact indicators pertain to such parameters as the helical pitch, projection data space z filter, image space z filter, projection data view weighting and slice thickness, and the noise indicators also pertain to the x-ray tube amperage in addition to those parameters.

In order to translate the picture quality levels during acceleration and deceleration in variable-pitch helical scanning into such picture quality indicators as the noise indicators and the artifact indicators in FIG. 31, projection data space z filter coefficients VZsXX and VZFXX and image space z filter coefficients IZsXX and IZfXX are prescribed for each helical pitch during acceleration or deceleration. XX therein represents the reference number of the coefficient.

Examples of projection data space z filter coefficients VZs and VZf refer to processing represented by (Formula 2) and (Formula 3) shown at z filter convolution of step S4 in FIG. 5.

A conceptual illustration of projection data space z filter convolution is given in FIG. 24. It is processing to convolute a weighting coefficient varying in the row direction (z direction) on projection data expanding in the channel direction and the row direction in each view, and to apply this to all the views. This enables the beam width ofthe projection data of each detector row in the row direction (z direction). In particular where a deconvolution filter is used, the beam width in the row direction (z direction) can be narrowed.

Examples of image space z filter coefficients IZs and IZf refer to processing represented by (Formula 8) and (Formula 9) shown at image space z filter convolution of step S7 in FIG. 5.

A conceptual illustration of image space z filter convolution is given in FIG. 25. In tomograms having undergone consecutive image reconstruction in the z direction, a weighting coefficient varying in the row direction (z direction) is convolute on each pixel of each such tomogram and nearby tomograms. This processing is applied to all the tomograms consecutive in the z direction.

This enables the slice thickness of each tomogram to be controlled. In particular where a deconvolution filter is used, the slice thickness can be reduced.

In this way, the picture quality can be optimized by controlling the projection data space z-directional filter coefficient and the image space z-directional filter coefficient for each scanning condition.

For instance, in the picture quality-prioritized mode, the picture quality can be optimized by controlling the projection data space z-directional filter coefficient and the image space z-directional filter coefficient for each of the indicators regarding the picture quality characteristics including, for instance, artifacts and image noise at each helical pitch.

Incidentally, the picture quality can be kept at the optimum by adjusting these projection data space z filter coefficients IZXX and image space z filter coefficients VZXX by using tomograms of a phantom or standard subject in advance.

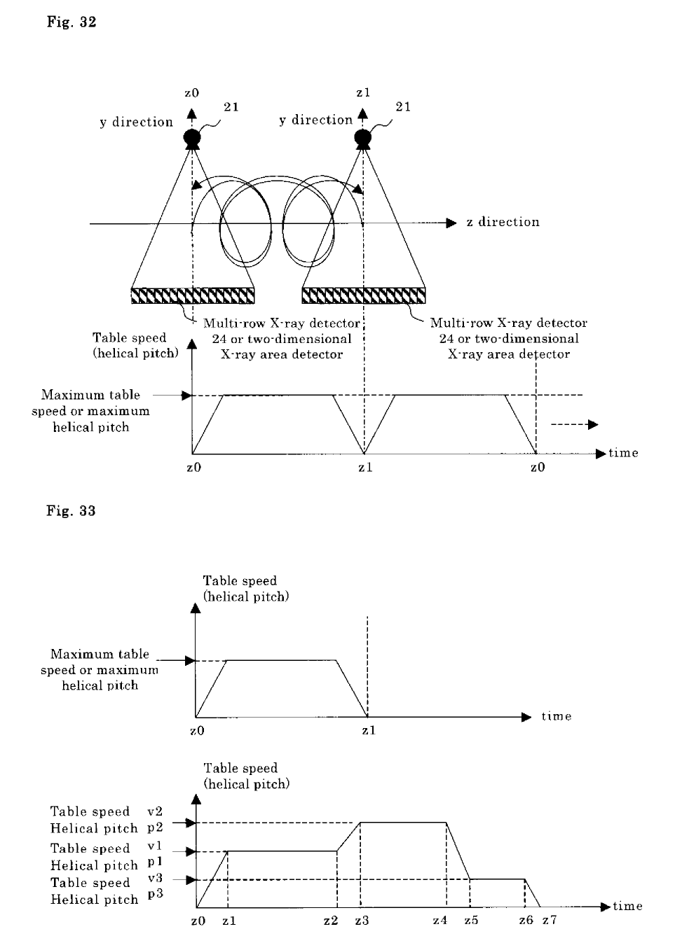

To add, the shuttle mode variable-pitch helical scanning is used for checking perfusion or the like in a scanning mode in which variable-pitch helical scanning is repeated a plurality of times while accelerating or decelerating in a certain range [z0, z1] of z-directional coordinates as shown in FIG. 32.

Unlike this, normal variable-pitch helical scanning is a scanning mode in which scanning is performed while accelerating or decelerating to vary the helical pitch in a certain range [z0, z1] of z-directional coordinates as shown in FIG. 33.

On the other hand, there are cases in which variable-pitch helical scanning is performed, as a developed form of the foregoing, in a range [z0, z7] of z-directional coordinates,

helical scanning being performed each at a constant speed, at a table speed v1 and a helical pitch p1 in a range [z1, Z2] of z-directional coordinates, at a table speed v2 and a helical pitch p2 in a range [z3, z4] of z-directional coordinates, and at a table speed v3 and a helical pitch p3 in a range [z5, z6] of z-directional coordinates:

accelerating in the z-directional coordinate range [z0, z1];

accelerating in the z-directional coordinate range [z2, z3];

decelerating in the z-directional coordinate range [z4, z5]; and

decelerating in the z-directional coordinate range [z6, z7]. This is particularly effective where high speed helical scanning of a plurality or organs or a plurality of subject regions is desired.

By the method of controlling the slice thickness described above, the whole range R0 of imaging variable-pitch helical scanning can be image-reconstructed at the same slice thickness as shown in FIG. 37.

Similarly, image reconstruction at slice thickness varied for different regions or different interest areas can also be achieved, at different slice thicknesses for R1, R2, R3 and R4.

### IMPLEMENTATION EXAMPLE 3

In Implementation Example 1 or Implementation Example 2, z coordinates at each time point are predicted as shown in the graph of FIG. 21 or FIG. 23. Or z-directional coordinate positions are measured with an encoder or the like provided on the scanning table 10 or the cradle 12 and, in extracting x-ray projection data in FIG. 10 at the time of three-dimensional image reconstruction for measuring the z-directional coordinate position of each view or views at fixed intervals, accurate three-dimensional back projection can be accomplished with the z-directional coordinate position of each view or views at fixed intervals figured out from these predicted or measured views being taken into consideration.

This makes available tomograms of high picture quality, uniform in picture quality in the z direction and relatively free from artifacts.

### IMPLEMENTATION EXAMPLE 4

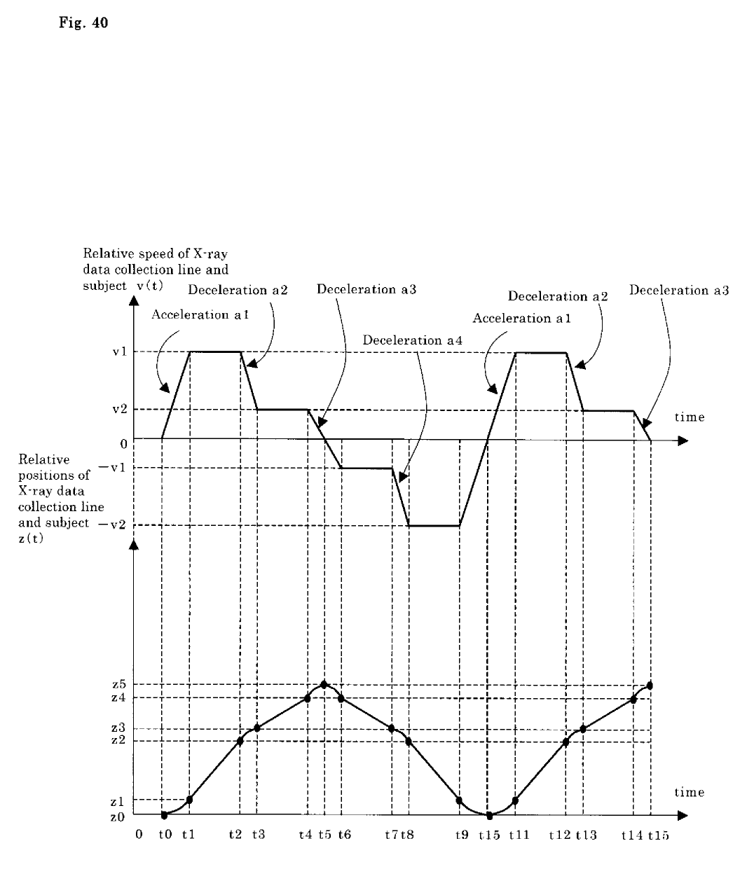

Implementation Example 3 represented a case in which tomograms of high picture quality, uniform in picture quality in the z direction and relatively free from artifacts are obtained by accurate three-dimensional back projection ofthree-dimensional image reconstruction by measuring or predicting the z-directional coordinate position of each view or views at fixed intervals. Similarly in the case of two-way variable-pitch helical scanning, tomograms of high picture quality, uniform in picture quality in the z direction and relatively free from artifacts, can be obtained. FIG. 40 shows the relative positions and relative speed of the x-ray data collection line and the subject in two-way variable-pitch helical scanning. The following description refers to the operation of a 1.5-round equivalent of two-way variable-pitch helical scanning.

X-ray data collection is started a little before the time point t0.

In the range of time points [t0, t1], movement is between z-directional coordinates [z0, z1] at an acceleration al and an initial speed 0.

In the range of time points [t1, t2], movement is between z-directional coordinates [z1, z2] at an acceleration 0 and a constant speed v1.

In the range of time points [t2, t3], movement is between z-directional coordinates [z2, z3] at a deceleration a2 and an initial speed v1.

In the range of time points [t3, t4], movement is between z-directional coordinates [z3, z4]; at an acceleration 0 and a constant speed v2.

In the range of time points [t4, t5], movement is between z-directional coordinates [z4, z5] at a deceleration a3 and an initial speed v2.

In the range of time points [t5, t6], movement is between z-directional coordinates [z5, z4] at a deceleration a3 and an initial speed 0;

In the range of time points [t6, t7] movement is between z-directional coordinates [z4, z3] at an acceleration 0 and a constant speed—v1;

In the range of time points [t7, t8], movement is between z-directional coordinates [z3, z2] at a deceleration a4 and an initial speed—v1.

In the range of time points [t8, t9], movement is between z-directional coordinates [z2, z1] at an acceleration 0 and a constant speed—v2;

In the range of time points [t9, t10], movement is between z-directional coordinates [z1, z0] at an acceleration a1 and an initial speed—v2;

In the range of time points [t10, t11], movement is between z-directional coordinates [z0, z1] at an acceleration al and an initial speed 0.

In the range of time points [t11, t12], movement is between z-directional coordinates [z1, z2] at an acceleration 0 and a constant speed v1;

In the range of time points [t12, t13], movement is between z-directional coordinates [z2, z3] at a deceleration a2 and an initial speed v1;

In the range of time points [t13, t14], movement is between z-directional coordinates [z3, z4] at an acceleration 0 and a constant speed v2.

In the range of time points [t14, t1], movement is between z-directional coordinates [z4, z5] at a deceleration a3 and an initial speed v2;

After time point t15, x-ray data collection is ended.

By performing two-way variable-pitch helical scanning in this way, a time series of three-dimensional images comprising tomograms consecutive in the z direction in the z-directional coordinate range of [z0, Z5] can be obtained.

In the above-described case, a three-dimensional image of [t0, t5], a three-dimensional image of [t5, t10] and a three-dimensional image of [t10, t15] are obtained as a time series of three-dimensional image. By measuring or predicting the z-directional coordinate position of each view or views at fixed intervals and accurately performing three-dimensional back projection of three-dimensional image reconstruction, positional deviations between forward and backward legs of images of two-way imaged variable-pitch helical scanning can be reduced. Especially, cine-displaying of three-dimensional images is accomplished from a three-dimensional image of [t0, t5] to a three-dimensional image of [t5, t10] to a three-dimensional image of [t10, t15] can be performed without perceivable positional deviations.

### IMPLEMENTATION EXAMPLE 5

With reference to Implementation Example 4, a method of picking up a time series of three-dimensional imaging by two-way variable-pitch helical scanning has been described. It is further possible, as an adaptation of this method, to apply the present invention to perfusion measurement, which was accomplished by using a time series of two-dimensional images by conventional cine-scanning.

A time series of three-dimensional images picked up by two-way variable-pitch helical scanning can be subjected to three-dimensional perfusion measurement. This enables the three-dimensional distribution of blood flows to be grasped.

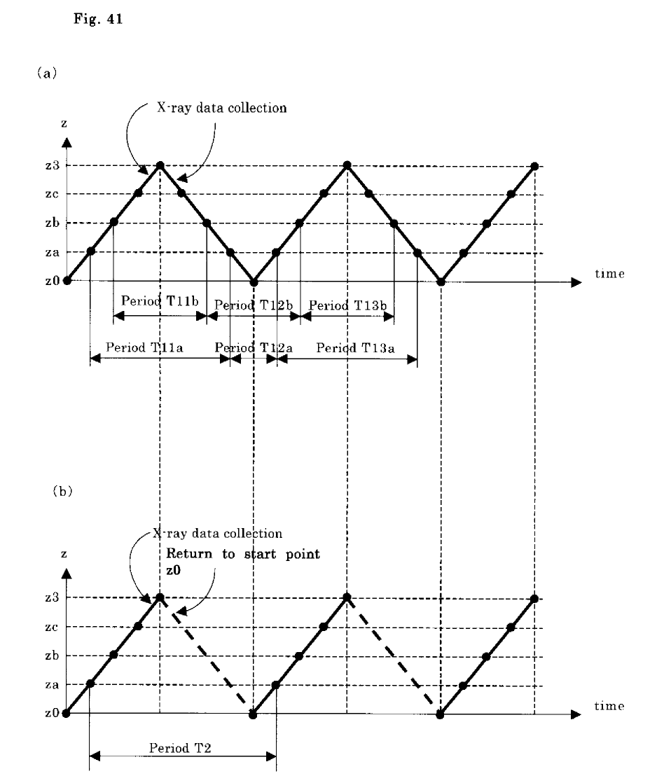

In the case of variable-pitch helical scanning by one-way repetition shown in FIG. 41(b), the time resolution is constant in a period T2 in the z-directional coordinate positions z0, za, zb, zc and z3. For this reason, a similar calculation method to the conventional perfusion measurement by a time series of two-dimensional images can be applied.

However, in the case of two-way variable-pitch helical scanning shown in FIG. 41(a), the time resolution is T11a, T12a, T11a and T12a at z9 in the z-directional coordinate positions z0, za, zb, zc and z3; the time resolution being uneven, sometimes long but short at other times.

However, at zb (provided that zb=(z0+z3)/2 is supposed), T11b=T12b=T13b holds, with a constant time resolution achieved at T11b. Thus, in two-way helical shuttle scanning, as the time resolution of images is sometimes variable depending on the z-directional coordinate position, perfusion measurement requires caution.

Incidentally, in a one-way leg of variable-pitch helical scanning as shown in FIG. 41(a) and FIG. 41(b), essentially the z-directional coordinate positions at different time points t are not linear, but curvilinear as shown in FIG. 40, but it is simplified to a straight in this illustration.

### IMPLEMENTATION EXAMPLE 6

Generally, in helical shuttle scanning and two-way variable-pitch helical scanning back and forth in the z direction, as it is a scanning processing consisting of accelerating parts, decelerating parts and constant speed parts of different speeds or one speed, trying to keep the picture quality of tomograms constant in the z direction would necessitate an automatic exposure mechanism for the x-ray CT apparatus.

Regarding this mode for carrying out the invention, optimization of the x-ray tube current taking into account the helical pitch in variable-pitch helical scanning or helical shuttle scanning back and forth in the z direction in an x-ray CT apparatus having an automatic exposure mechanism, and variations in the number of revolutions of projection data for image reconstruction will be discussed below.

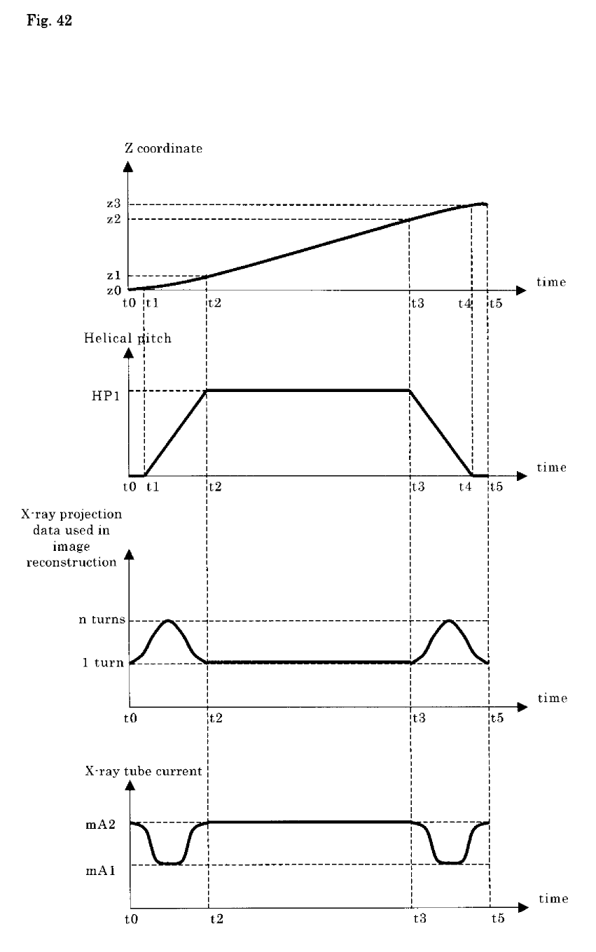

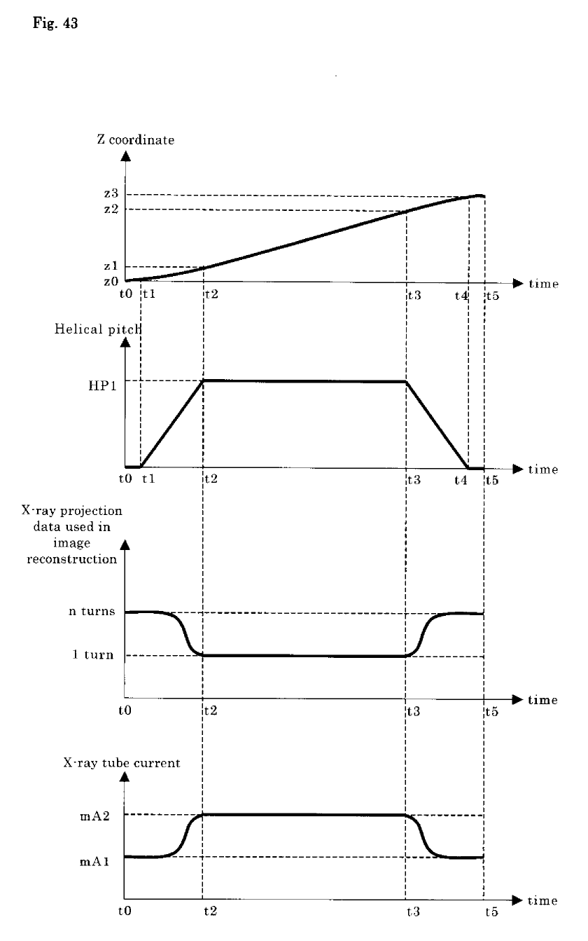

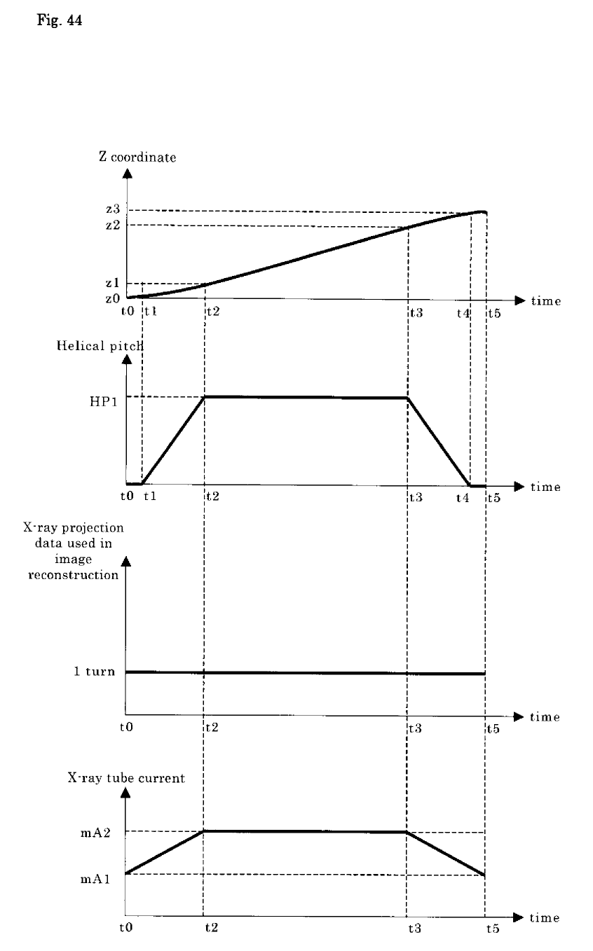

As shown in FIG. 42, FIG. 43 and FIG. 44, in variable-pitch helical scanning or helical shuttle scanning back and forth in the z direction, the helical pitch varies with the z direction or the direction of time points t. In the relative actions of the subject and the x-ray data collection line, the helical pitch becomes 0 in particular at the start point z0 and the stop point z3. Thus, in some cases, the cradle 12 or the scanning table 10 mounted with the subject of the x-ray data collection line stands still for a certain length of time in the relative actions between the subject and the x-ray data collection line at the start point z0 or the stop point z3. Also, the S/N ratio can be improved by using x-ray projection data for use in image reconstruction for more than one turn at the time of acceleration or deceleration of the cradle 12 or the scanning table 10 mounted with the subject or x-ray data collection line.

In variable-pitch helical scanning or helical shuttle scanning back and forth in the z direction, shown in FIG. 42, the z coordinates are controlled in the following way.

The x-ray data collection line as viewed from the subject between the time points [t0, t1] stands still at z0.

The x-ray data collection line as viewed from the subject between the time points [t1, t2] moves between [z0, z1] under acceleration.

The x-ray data collection line as viewed from the subject between the time points [t2, t3] moves between [z1, z2] at a constant speed.

The x-ray data collection line as viewed from the subject between the time points [t3, t4] moves between [z2, z3] under deceleration.

The x-ray data collection line as viewed from the subject between the time points [t4, t5] stands still at z3.

The helical pitch is controlled in the following way.

It is 0 between the time points [t0, t1].

It is accelerated between the time points [t1, t2].

It becomes constant at a helical pitch HP1 between the time points [t2, t3].

It is decelerated between the time points [t3, t4].

It returns to 0 between the time points [t4, t5].

The x-ray projection data for use in image reconstruction controlled in the following way, provided that n>1 holds as indicated in FIG. 42.

They undergo one turn at the time point t0.

X-ray projection data of the maximum value n turns are used on the way between the time points [t0, t2].

They return to one turn at the time point t2.

They are constant at one turn between the time points [t2, t3].

They undergo one turn at the time point t3, but x-ray projection data of the maximum value n turns are used on the way between the time points [t3, t5].

They return to one turn at the time point t5.

Especially in the parts where the helical pitch is 1 or less, the range of x-ray projection data for use in image reconstruction can be broader, which contributes to picture quality improvement. This proves particularly effective in accelerating or decelerating helical shuttle scanning and variable-pitch helical scanning back and forth in the z direction.

In this case, the x-ray projection data for use in image reconstruction are subjected to one turn between the time points [t0, t5] and between the time points [t2, t3], to bring it closer to image reconstruction by usual conventional scanning (axial scanning) between the time points [t0, t5] and to bring it closer to image reconstruction by helical scanning between the time points [t2, t3].

For this reason, considering the control of the x-ray tube current to keep the picture quality uniform between the time points [t0, t4], the x-ray tube current is controlled as indicated in FIG. 42, provided that mA2>mA1 holds.

At the time point t0, the x-ray tube current is mA2.

On the way between the time points [t0, t2], the x-ray tube current is brought down to its minimum mA1.

At the time point t2, it returns to mA2.

Between the time points [t2, t3], the x-ray tube current is constant at mA2.

At the time point t3, the minimum x-ray tube current is mA2.

Between the time points [t3, t5], the minimum x-ray tube current mA1 is used.

At the time point t5, the x-ray tube current returns to mA2.

Incidentally, between the time points [t0, t2] and between the time points [t3, t5], controlling the relationship among the helical pitch HP, the x-ray tube current mA and the length L of the range of x-ray projection data for use in image reconstruction according to (Formula 22) below can give a constant level of picture quality in the z direction.

$$\begin{matrix}[ Mathematical Expression 18 ] & \\ la / ( la + lb ) \frac{mA \cdot L}{HP} Const ( Constant ) & ( Formula 22 )\end{matrix}$$

Thus, by so controlling the ratio between the product of the x-ray tube current mA and the length L of the range of x-ray projection data and the helical pitch HP as to keep it constant or substantially constant, a constant level of picture quality in the z direction can be obtained.

In the variable-pitch helical scanning or helical shuttle scanning back and forth in the z direction illustrated in FIG. 43, the z coordinates of the x-ray data collection line as viewed from the subject are controlled in the following way.

The x-ray data collection line as viewed from the subject between the time points [t0, t1] stands still at z0.

The x-ray data collection line as viewed from the subject between the time points [t1, t2] moves between [z0, z1] under acceleration.

The x-ray data collection line as viewed from the subject between the time points [t2, t3] moves between [z1, z2] at a constant speed.

The x-ray data collection line as viewed from the subject between the time points [t3, t4] moves between [z2, z3] under deceleration.

The x-ray data collection line as viewed from the subject between the time points [t4, t5] stands still at z3.

The helical pitch is controlled in the following way.

Between the time points [t0, t1], it is 0.

Between the time points [t1, t2], it is accelerated.

Between the time points [t2, t3] it is constant at the helical pitch HP1.

Between the time points [t3, t4], it decelerates.

Between the time points [t4, t5], it returns to 0.

The x-ray data collection line for use in image reconstruction are controlled in the following way, provided that n>1.

Between the time points [t0, t2], they decrease from n turns to one turn.

Between the time points [t2, t3], they are constant at 1 turn.

Between the time points [t3, t4], they increase from one turn to n turns.

For this reason, more x-ray projection data are used between the time points [t0, t2] and between the time points [t3, t4], and the picture quality is improved. Therefore, with a view to keeping the picture quality constant between the time points [t0, t4], the x-ray tube current can be reduced between the time points [t0, t2] and between the time points [t3, t4]. Especially in the parts where the helical pitch is 1 or less, the range of x-ray projection data for use in image reconstruction can be broader, which contributes to picture quality improvement. This proves particularly effective in accelerating or decelerating helical shuttle scanning and variable-pitch helical scanning.

For this reason, it is intended to so control the x-ray tube current as to keep the picture quality constant between the time points [t0, t4]. The x-ray tube current is controlled as indicated in FIG. 43, provided that mA2>mA1.

At the time point 10, it is the x-ray tube current mA1.

Between the time points [t0, t2], there is an increase foam the x-ray tube current mA1 to the x-ray tube current mA2.

### At the time point t2, it becomes the x-ray tube current mA2.

Between the time points [t2, t3], it is constant at the x-ray tube current mA2′. At the time point t3, it is the x-ray tube current mA2.

Between the time points [t3, t5], there is a decrease from the x-ray tube current mA2 to the x-ray tube current mA1.

At the time point t5, it returns to the x-raytube current mA1.

Incidentally, between the time points [t0, t2] and between the time points [t3, t5], controlling the relationship among the helical pitch HP, the x-ray tube current mA and the length L of the range of x-ray projection data for use in image reconstruction according to (Formula 22) stated above gives a constant level of picture quality in the z direction.

Thus, by so controlling the ratio between the product of the x-ray tube current mA and the length L of the range of x-ray projection data and the helical pitch HP as to keep it constant or substantially constant, a constant level of picture quality in the z direction can be obtained.

In this case, in order to it closer to image reconstruction by nonnal helical scanning between the time points [t2, t3], the projection data for use in image reconstruction are rotated by one turn between the time points [t2, t3]. Between the time points [t0, t2] and between the time points [t3, t5], the speed of advancing in the z direction as the relative speed between the scanning table and the data collection line slows down as they approach the time point t0 and time point t5.

For this reason, improvement with respect to image noise is accomplished with increasing the slice thickness, which is the thickness of the tomogram in the z direction, namely without sacrificing the resolution of the tomogram in the z direction It is intended thereby to lower the x-ray tube current and reduce exposure to x-rays. For this reason, x-ray projection data of n turns are used for image reconstruction at the time point t0 and the time point t5.

In the variable-pitch helical scanning or helical shuttle scanning illustrated in FIG. 44, the z coordinates are controlled in the following way.

The x-ray data collection line as viewed from the subject between the time points [t0, t1] stands still at z0.

The x-ray data collection line as viewed from the subject between the time points [t1, t2] moves between [z0, z1] under acceleration.

The x-ray data collection line as viewed from the subject between the time points[t2, t3] moves between [z1, z2] at a constant speeds.

The x-ray data collection line as viewed from the subject between the time points [t3, t4] moves between [z2, z3] under acceleration.

The x-ray data collection line as viewed from the subject between the time points [t4, t5] stands still at z3.

The helical pitch is controlled in the following way.

Between the time points[t0, t1], it is 0.

Between the time points [t1, t2], it accelerates.

Between the time points [t2, t3], it becomes constant at a helical pitch HP1.

Between the time points [t3, t4], it decelerates.

Between the time points [t4, t5], it returns to 0.

The x-ray projection data for use in image reconstruction are kept constant and rotated by one turn between the time points [t0, t5]. In this case, priority is given to keeping the time resolution of tomogram constant, and the x-ray projection data for use are kept constant.

For this reason, it is considered to so control the x-ray tube current as to keep the picture quality constant between the time points [t0, t4]. The x-ray tube current is controlled as shown in FIG. 44, provided that mA2>mA1 holds.

At the time point t0, it is the x-ray tube current mA1.

Between the time points [t0, t2], there is an increase foam the x-ray tube current mA1 to the x-ray tube current mA2. Incidentally, if the helical pitch increases then, the x-ray tube current will also increase. It is advisable to so effect control as to keep the ratio between the helical pitch and the x-ray tube current constant or substantially constant.

At the time point t2, it becomes the x-ray tube current mA2.

Between the time points [t2, t3], it is constantly the x-ray tube current mA2.

At the time point t3, it is the x-ray tube current mA2.

Between the time points [t3, t5], there is a decrease from the x-ray tube current mA2 to the x-ray tube current mA1. Incidentally, if the helical pitch decreases then, the x-ray tube current will also decrease. It is advisable to so effect control as to keep the ratio between the helical pitch and the x-ray tube current constant or substantially constant.

At the time point t5, it returns to the x-ray tube current mA1.

In this way, control has been so attempted as to bring the picture quality of tomograms to normal conventional scanning and helical scanning as illustrated in FIG. 42. The control illustrated in FIG. 43 is intended to reduce exposure to x-rays during acceleration and deceleration without sacrificing the picture quality of tomograms. The control illustrated in FIG. 44 is intended to keep the time resolution of tomograms constant.

In these cases, the top priority in control was given to the control of the helical pitch, which is the variable of the picture quality of tomograms, and the variables of data quantity used in image reconstruction, followed by the control of the x-ray tube current. In this way, with a view to compatibility with the variation table of the x-ray tube current in the z direction obtained from scout images, instead of first using the x-ray tube current, which is a variable for controlling the picture quality of tomograms, other variables for controlling the picture quality were controlled with priority, and the variation table of the x-ray tube current in the z direction obtained from scout images was corrected by controlling those variables. It is possible realize an automatic exposure function for the x-ray CT apparatus by controlling the x-ray tube current after that.

The flow of processing in the above-described mode for implementation illustrated in FIG. 42, FIG. 43 and FIG. 44 is traced below.

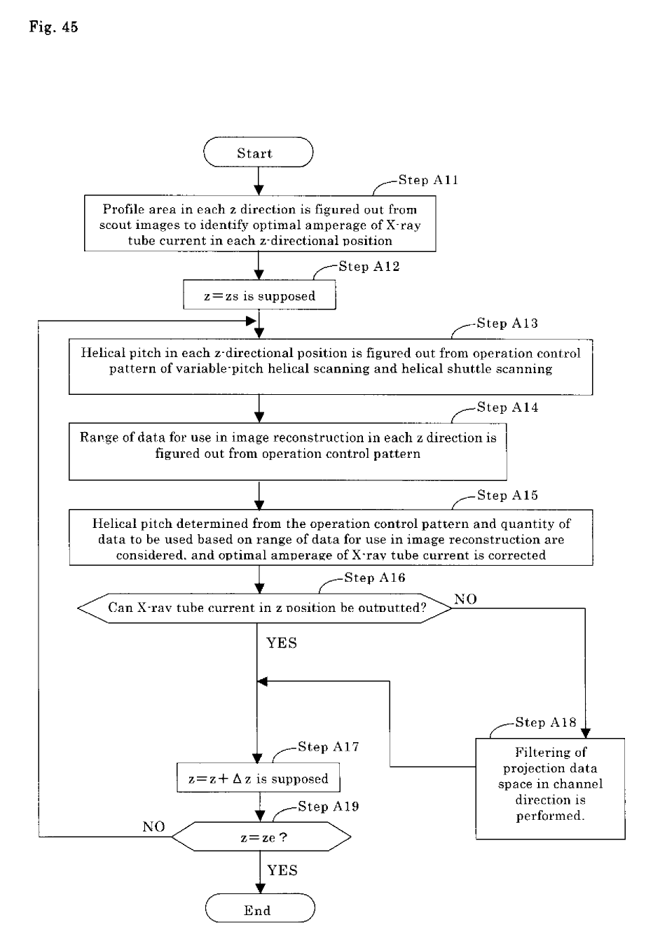

The variable-pitch helical scanning or helical shuttle scanning shown in FIG. 42, FIG. 43 and FIG. 44 is controlled in the flow of processing charted in FIG. 45.

At step A11, the profile area in each z direction is figured out from scout images to identify the optimal amperage of the x-ray tube current in each z-directional position.

At step A12, z=zs is supposed, provided that zs is the starting coordinate in the z direction.

At step A13, the helical pitch in each z-directional position is figured out from the operation control pattern of the variable-pitch helical scanning and helical shuttle scanning.

At step A14, the range of data for use in image reconstruction in each z direction is figured out from the operation control pattern.

At step A15, the helical pitch determined from the operation control pattern and the quantity of data to be used based on the range of data for use in image reconstruction are considered, and the optimal amperage of the x-ray tube current is corrected accordingly.

At step A16, it is judged whether or not the x-ray tube current in the z position can be outputted and, if YES, the processing will advance to step A17 or, if NO, to step A18.

At step A17, z=z+Δz is supposed.

At step A18, filtering of projection data space in the channel direction is performed.

At step A19, it is judged whether or not z is equal to or larger than ze and, if z is equal to or larger than ze, that is YES, the processing is completed or, if z is not equal to or not larger than ze, that is NO, it returns to step A13, provided that the z-directional terminal coordinate is ze.

Incidentally, in the above-described case, the use of the helical pitch and other picture quality variables than the length of range used by the x-ray projection data in image reconstruction as the picture quality variables of tomograms to be used with priority over the x-ray tube current could provide a similar effect.

In the x-ray CT apparatus 100, the x-ray CT apparatus or the x-ray CT imaging method according to the invention provide the effect of reducing exposure in conventional scanning (axial scanning) or cine-scanning or helical scanning to the x-ray cone beam expanding in the z direction existing at the time of starting and ending the conventional scanning (axial scanning) or cine-scanning or helical scanning by the x-ray CT apparatus having a conventional multi-row x-ray detector or a two-dimensional x-ray detector, represented by a flat panel x-ray detector.

Incidentally, the image reconstruction method in this embodiment may be the usual three-dimensional image reconstruction method according to the already known Feldkamp method. It may even be some other three-dimensional image reconstructing method.

Also, a uniform slice thickness from row to row and picture quality in terms of artifacts and noise are achieved in this embodiment by convoluting row-directional (z-directional) filters differing in coefficient from row to row thereby to adjust fluctuations in picture quality due to differences in x-ray cone angle, and various z-directional filter coefficients are conceivable for this purpose, any of which can give a similar effect.

Although this embodiment has been described under the assumption of using the x-ray CT apparatus for medical purposes, it can as well be utilized as an x-ray CT apparatus for industrial purposes or an x-ray CT-PET apparatus or an x-ray CT-SPECT apparatus in combination with some other apparatus.

Whereas the optimization of the projection data space z filter coefficient and the image space z filter coefficient in this embodiment was touched upon in FIG. 31 with respect to the case of variable-pitch helical scanning, actually various ways of optimization are actually conceivable depending on differences in processing time, picture quality and slice thickness targets, other cases of conventional scanning (axial scanning) or cine-scanning or helical scanning or helical shuttle scanning can be expected to provide similar effects.

1. An x-ray CT apparatus comprising:
an x-ray data collecting device for collecting x-ray projection data transmitted by a subject positioned between an x-ray generating device and a multi-row x-ray detector, while rotating said x-ray generating device and said multi-row x-ray detector around a rotation center positioned in-between;
an image reconstructing device for performing image reconstruction from the projection data collected from said x-ray data collecting device;
an image display device for displaying a tomogram obtained by image reconstruction; and
a scanning condition setting device for setting various scanning conditions of tomography scanning,
wherein said x-ray data collecting device is operable for variable-pitch helical scanning in which x-ray projection data of the subject on a scanning table is collected by moving the scanning table while varying the speed relative to a scanning gantry in a z direction perpendicular to an xy plane which is the rotating plane of the x-ray generating device and the two-dimensional x-ray area detector, and of which starting of the x-ray data collection and starting of the scanning table movement relative to the scanning gantry and/or stopping of the x-ray data collection and stopping of the scanning table movement relative to the scanning gantry are asynchronously executed.

2. An x-ray CT apparatus according to claim 1, wherein said x-ray data collecting device is operable for said variable-pitch helical scanning of which starting the collection of x-ray data is executed after starting of the scanning table movement relative to the scanning gantry.

3. An x-ray CT apparatus according to claim 1, wherein said x-ray data collecting device is operable for said variable-pitch helical scanning of which stopping of the movement of the scanning table relative to the scanning gantry is executed after stopping of the x-ray data collection.

4. An x-ray CT apparatus according to claim 1, wherein said x-ray data collecting device is operable for said variable-pitch helical scanning of which starting of the movement of the scanning table relative to the scanning gantry is executed after starting of the x-ray data collection.

5. An x-ray CT apparatus according to claim 1, wherein said x-ray data collecting device is operable for said variable-pitch helical scanning of which stopping the collection of x-ray data is executed after stopping of the scanning table movement relative to the scanning gantry.

6. The x-ray CT apparatus according to claim 4, wherein said collection of x-ray data is executed by rotating the rotary unit of the scanning gantry during a period in which the scanning table and the scanning gantry are at halt relative to each other.

7. The x-ray CT apparatus according to claim 5, wherein said collection of x-ray data is executed by rotating the rotary unit of the scanning gantry during a period in which the scanning table and the scanning gantry are at halt relative to each other.

8. The x-ray CT apparatus according to claim 6, wherein view angle at which the rotary unit of the scanning gantry rotates to collect x-ray data during the period in which the scanning table and the scanning gantry are at halt relative to each other is not less than the fan angle+180 degrees.

9. The x-ray CT apparatus according to claim 7, wherein view angle at which the rotary unit of the scanning gantry rotates to collect x-ray data during the period in which the scanning table and the scanning gantry are at halt relative to each other is not less than the fan angle+180 degrees.

10. The x-ray CT apparatus according to claim 1, wherein said image reconstructing device is configured to perform image reconstruction of the whole imaging range in the same slice thickness.

11. The x-ray CT apparatus according to claim 1, wherein said image reconstructing device is configured to perform image reconstruction in the same slice thickness within a range of the number of ranges into which the whole imaging range is divided.

12. The x-ray CT apparatus according to claim 1, wherein said image reconstructing device is configured to control the slice thickness by performing filter convolution in the z direction (row direction).

13. The x-ray CT apparatus according to claim 1, wherein said image reconstructing device is configured to control the slice thickness by multiplying the projection data of each view by a weighting coefficient.

14. The x-ray CT apparatus according to claim 13, wherein said image reconstructing device is configured to use projection data of not less than 360 degrees as the projection data.

15. The x-ray CT apparatus according to claim 1, wherein said image reconstructing device is configured to control the slice thickness by weighted addition by multiplying image-reconstructed tomograms consecutive in the z direction by a weighting coefficient.

16. The x-ray CT apparatus according to claim 1, wherein said x-ray data collecting device includes the scanning gantry which performs variable-pitch helical scanning at an inclination to the xy plane.

17. The x-ray CT apparatus according to claim 1, wherein said x-ray data collecting device includes a planar x-ray detector or an x-ray detector combining a plurality of planar x-ray detectors.

18. The x-ray CT apparatus according to claim 1, wherein:
said x-ray data collecting device is operable for measuring z-directional coordinate position of at least one view, and
said reconstructing device is operable for reconstructing using a measured value of the z-directional coordinate position of at least one view or a predicted value of the z-directional coordinate position of at least one view.

19. The x-ray CT apparatus according to claim 1, wherein:
said x-ray data collecting device is operable for consecutively repeating x-ray data collection in a certain range of z-directional coordinate positions.

20. A method comprising changing a helical pitch during z-direction velocity changes of a moving gantry to obtain substantially uniform image quality in a plurality of reconstructed images.
